SEQUENCE LISTING (17 sequences)

SEQ ID NO: 1 - DNA, 29 bp, synthetic construct
  source 1..29: mol_type = other DNA
agtccgtggtagggcaggttggggtgact

SEQ ID NO: 2 - DNA, 36 bp, synthetic construct
  source 1..36: mol_type = other DNA
aaaaaaaagtccgtggtagggcaggttggggtgact

SEQ ID NO: 3 - DNA, 29 bp, synthetic construct
  source 1..29: mol_type = other DNA
agtccgtaataaagcaggttaaaatgact

SEQ ID NO: 4 - DNA, 36 bp, synthetic construct
  source 1..36: mol_type = other DNA
agtcaccccaacctgccctaccacggactttttttt

SEQ ID NO: 5 - DNA, 30 bp, synthetic construct
  source 1..30: mol_type = other DNA
agtccgtggtagggcaggttggggtgactt

SEQ ID NO: 6 - DNA, 31 bp, synthetic construct
  source 1..31: mol_type = other DNA
agtccgtggtagggcaggttggggtgacttt

SEQ ID NO: 7 - DNA, 32 bp, synthetic construct
  source 1..32: mol_type = other DNA
agtccgtggtagggcaggttggggtgactttt

SEQ ID NO: 8 - DNA, 33 bp, synthetic construct
  source 1..33: mol_type = other DNA
aaaaagtcaccccaacctgccctaccacggact

SEQ ID NO: 9 - RNA, 37 nt, synthetic construct
  source 1..37: mol_type = other RNA
  modified_base 37: mod_base = OTHER; note = Inverted dT
gagagccccagcgagataatacttggccccgctcttt

SEQ ID NO: 10 - RNA, 57 nt, synthetic construct
  source 1..57: mol_type = other RNA
  modified_base 57: mod_base = OTHER; note = Inverted dT
aaaaaaaaaaaaaaaaaaaagagagccccagcgagataatacttggccccgctcttt

SEQ ID NO: 11 - DNA, 41 bp, synthetic construct
  source 1..41: mol_type = other DNA
tattatctcgctggggctctctttttttttttttttttttt

SEQ ID NO: 12 - RNA, 26 nt, synthetic construct
  source 1..26: mol_type = other RNA
  modified_base 26: mod_base = OTHER; note = Inverted dT
gggaacaaagctgaagtacttaccct

SEQ ID NO: 13 - DNA, 35 bp, synthetic construct
  source 1..35: mol_type = other DNA
ttctgctttgttccctttttttttttttttttttt

SEQ ID NO: 14 - RNA, 46 nt, synthetic construct
  source 1..46: mol_type = other RNA
  modified_base 46: mod_base = OTHER; note = Inverted dT
aaaaaaaaaaaaaaaaaaaagggaacaaagctgaagtacttaccct

SEQ ID NO: 15 - DNA, 15 bp, synthetic construct
  source 1..15: mol_type = other DNA
ggttggtgtggttgg

SEQ ID NO: 16 - DNA, 35 bp, synthetic construct
  source 1..35: mol_type = other DNA
aaaaaaaaaaaaaaaaaaaaggttggtgtggttgg

SEQ ID NO: 17 - DNA, 35 bp, synthetic construct
  source 1..35: mol_type = other DNA
ttttttttttttttttttttccaaccacaccaacc

(19) United States
(12) Patent Application Publication
(10) Pub. No.: US 2026/0176636 A1
(43) Pub. Date: Jun. 25, 2026

(54) APTAMER-DIRECT INHIBITOR CONJUGATES AND METHODS OF USING THE SAME

(71) Applicant: Duke University, Durham, NC (US)

(72) Inventors: Bruce SULLENGER, Durham, NC (US); Haixiang YU, Durham, NC (US)

(21) Appl. No.: 19/126,958

(22) PCT Filed: Nov. 2, 2023
(86) PCT No.: PCT/US2023/078453

§ 371 Date: May 2, 2025

Related U.S. Application Data
(60) Provisional application No. 63/421,756, filed on Nov. 2, 2022.

(51) Int. Cl. C12N 15/115; A61K 48/00; C12N 15/113

(52) U.S. Cl. CPC C12N 15/115; A61K 48/00; C12N 15/113; C12N 2310/3521

(57) ABSTRACT

Described herein are compositions for inhibiting a protease or coagulation target comprising a nucleic acid aptamer covalently linked to a small molecule inhibitor and methods for using the same to inhibit coagulation. Also provided are methods of reversing the anti-coagulation effects of the composition by administering an antidote.

### CROSS-REFERENCE TO RELATED APPLICATIONS

This application claims priority to U.S. Provisional Application No. 63/421,756 filed on Nov. 2, 2022, the contents of which are incorporated by reference in their entireties.

### STATEMENT REGARDING FEDERALLY SPONSORED RESEARCH

This invention was made with government support under 5P01-HL139420 awarded by the National Institutes of Health. The government has certain rights in the invention.

### REFERENCE TO AN ELECTRONIC SEQUENCE LISTING

The contents of the electronic sequence listing (155554.00720.xml; Size: 18,243 bytes; and Date of Creation: Nov. 1, 2023) are herein incorporated by reference in its entirety.

### BACKGROUND OF THE INVENTION

Fibrin blood clot formation is mediated by a series of enzymatic reactions that occur on cellular and vascular surfaces. The coagulation proteins circulate in the blood as inactive proteins (zymogens) and upon stimulation are proteolyzed to generate active enzymes. Traditional coagulation models represent the series of reactions as a Y-shaped “cascade” with two separate pathways—the extrinsic and intrinsic pathway—that ultimately converge into a final common pathway (Macfarlane, Nature 202:498-9 (1964), Davie and Ratnoff, Science 145:1310-2 (1964)). Thrombin is the final enzyme formed in the coagulation cascade, and the rate of thrombin formation, as well as the amount of thrombin formed directly influences fibrin clot stability and structure (Wolberg, Blood Rev 21:131-42 (2007)).

Inappropriate thrombin generation can result in pathological blood clot formation, termed thrombosis. The treatment of patients with thrombosis almost always includes the administration of an anticoagulant therapeutic to impair procoagulant protein function and prevent blood coagulation. Researchers currently debate the optimal therapeutic target as the degree of anticoagulation required varies depending on the clinical indication. For example, lower levels of anticoagulation are desired for prophylactic treatment of high-risk patients, while potent anticoagulation is required during surgical procedures, such as cardiopulmonary bypass (CPB), to treat thrombosis.

Genetic studies with knockout mice have been performed to study the role of each coagulation protein and pathway specific responses. Although Hemophilic mice (FVIII or FIX deficiency) have been extensively studied to discern the role of these proteins during in vivo coagulation, genetically null mice for TF, FVII, FX, and prothrombin are not viable, making similar studies unfeasible (Mackman, Arterioscier Thromb Vasc Biol 25: 2273-81 (2005)). Alternatively, inhibiting clotting proteins with currently available anticoagulant therapeutics can functionally remove the enzyme from the system and thereby clarify the role of these proteins in clot formation. Although small molecule anticoagulants have been generated toward a few coagulation enzymes (i.e., thrombin and FXa), it has been challenging to design similar compounds toward the upstream coagulation enzymes (i.e., FVIIa and FIXa). Thus, alternative classes of therapeutics that can be applied to inhibit all of the procoagulant proteins are needed to fully probe and directly compare the contributions of each pathway.

Aptamers, or single-stranded oligonucleotides, are nucleic acid (DNA or RNA) ligands that bind specifically to their therapeutic targets with high affinity. Aptamers possess a number of features that render them useful as therapeutic agents. They are relatively small (8 kDa to 15 kDa) synthetic compounds that possess high affinity and specificity for their target molecules (equilibrium dissociation constants ranging from, for example, 0.05-1000 nM). Thus, they embody the affinity properties of monoclonal antibodies and single chain antibodies (scFv's) with the chemical production properties of small peptides. Aptamers can be generated against target molecules, such as soluble coagulation proteins, including coagulation factor VIIa (FVIIa) (Layzer and Sullenger, Oligonucleotides 17:1-11 (2007)), factor IXa (FIXa) (Rusconi et al, Nature 419: 90-4 (2002)), factor X (FXa) (Buddai et al, J Bio Chem 285:52 12-23 (2010)), and prothrombin/thrombin (Layzer and Sullenger, Oligonucleotides 17:1-11 (2007), Bompiani et al, J Thromb Haemost 10:870-80 (2012)).

There is need in the art, however for methods and compositions of aptamer-directed inhibitor conjugates, which can bind a single target with high potency, and importantly, the activity of the aptamer-directed inhibitor can be rapidly and fully reversed.

### BRIEF SUMMARY OF THE INVENTION

Disclosed herein are compositions for inhibiting a protease or coagulation target comprising a nucleic acid aptamer covalently linked to a small molecule inhibitor and methods for using the same. In some embodiments the composition comprises a nucleic acid aptamer covalently linked to a small molecule inhibitor, wherein the nucleic acid aptamer targets the exosite of the proteases or the coagulation target and wherein the small molecule inhibitor inhibits the active site of the protease or the coagulation target. In some embodiments the protease or coagulation targets include VWF, FV11a, FIXa, FXa, prothrombin, and thrombin. In exemplary embodiments the compositions comprise aptamers HD22, 11f7t, Tog25 or HD1; the small molecule inhibitors include dabigatran or apixaban, and linkers are used. The linkers may compose nucleotide and non-nucleotide linkers and may vary in length, position relative to the aptamer and modifications.

Another aspect of the invention provides methods for inhibiting coagulation and for treating a subject in need of anti-coagulation therapy. The methods comprise contacting a site of coagulation or potential coagulation with a composition described herein, or administering the composition described herein to a subject in need for a period of time sufficient to allow a reduction in coagulation in the subject and optionally administering an oligonucleotide antidote to the aptamer, wherein the oligonucleotide antidote reverses the anti-coagulation activity of the composition.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. EXACT inhibitor HD22-7A-DAB potently inhibits thrombin. a-b, Crystal structures of hirudin-thrombin complex (a) in comparison to HD22-7A-DAB-thrombin complex (b). In both inhibitors, the active site-binding domain (magenta) and the exosite binding domain (green) are connected by a linker domain (grey), which allows synergistic binding to thrombin (tan). The portion of the linker region of HD22-7A-DAB that cannot be resolved was reconstructed by molecular modeling c, Thrombin inhibition by HD22-7A-DAB and other inhibitors in fluorogenic substrate cleavage assay. HD22-7A-DAB showed a significantly higher inhibitory effect of thrombin than DAB, HD22, or their equimolar mixture, and can be efficiently reversed by an antisense oligonucleotide (AO2). d, two-step binding model of EXACT inhibitor (AI) to protease (E) determined by dissociation constants K_{E,A}, K_{E,I}, and K_{EA,I}. e, Characterization of binding kinetics of HD22-7A-DAB and its derivatives to thrombin (1.6 nM) by bio-layer interferometry. f-g, The potency of HD22-DAB conjugates with different dA linker length was tested (f) and their maximum inhibition and IC₅₀ on thrombin were determined (g). h-i, Inhibition of factor Xa (h) and thrombin (i) activity by EXACT inhibitors constructed with factor Xa (11F7t) or thrombin (HD22)-binding aptamer, respectively, demonstrate that the selectivity of the small molecule inhibitor DAB can be dictated by the exosite-binding aptamer to which it is conjugated.

FIG. 2: Crystal structure of HD22-7A-DAB (HD22 aptamer-green and DAB-pink) binding to thrombin S195A (wheat) superpositioned with previously published HD22 (orange, PDBID 4I7Y) and dabigatran (cyan, PDBID 1KTS) structures binding to thrombin. The active site was zoomed in to show thrombin's interacting residues (red) with dabigatran.

FIG. 3: HD22-7A-DAB efficiently and reversibly inhibits multiple functions of thrombin. a-b, Inhibition of thrombin mediated fibrin clot formation by HD22-7A-DAB in a fibrinogen turbidity assay. a, Time-course of fibrin clot formation with 0.8 mg/mL fibrinogen and 2.5 nM thrombin in the absence (grey) or presence of thrombin inhibitors (50 nM) including HD22 (red), DAB (black), equimolar mixture of HD22 and DAB mixture (blue), and EXACT inhibitor HD22-7A-DAB (yellow) characterized by the absorbance at 550 nm. The kinetics of antidote reversal was characterized by adding 2 μM of AO2 to the reaction 10 min after fibrinogen addition (green). b, The lag time, maximum absorption, and time to reach 90% maximum absorption (t₉₀) in the absence and presence of different inhibitors were determined. c-d, Inhibition of thrombin (1 nM) mediated FVIII (100 nM) activation by EXACT inhibitor HD22-7A-DAB. c, PAGE analysis of thrombin-mediated FVIII cleavage the absence or presence of thrombin inhibitors (100 nM). d, Structure of human FVIII and thrombin cleavage site. e, Thrombin's activation of FVIII in the absence and presence of thrombin inhibitors were quantified using the time-course concentration of intact light chain (a3A3C1C2) determined by band intensity.

FIG. 4: Anticoagulation activity of EXACT inhibitor HD22-7A-DAB. a-c, Different concentrations of HD22, DAB, equimolar mixture of HD22 and AB, or HD22-7A-DAB were added to normal human plasma and incubated for 5 min at 37° C. and a, Thrombin time (TT), b, Prothrombin time (PT), and c, Partial thromboplastin time (aPTT) assays were performed to characterize anticoagulant efficacy. To characterize antidote reversal of HD22-7A-DAB, another 5-min incubation of antisense oligo AO2 (2 μM) with the plasma-HD22-7A-DAB mixture was performed. d. Anticoagulant activity of HD22-7A-DAB in whole blood was characterized using the active clotting time (ACT) assay and compared to no inhibitor (Ctrl), HD22, DAB, equimolar mixture of HD22 and DAB, and UFH (unfractionated heparin)-mediated anticoagulation. ACT exceeded the measurable range of the analyzer (999 sec) in the presence of 2 μM HD22-7A-DAB. *, P<0.05, ns is not significant.

FIG. 5: Synthesis and purification of the HD22-DAB conjugates. a, The HD22-DAB conjugates were synthesized from dabigatran and 5′ Amine modified HD22 derivatives via EDC/NHS conjugation following by (b) HPLC purification where the conjugated fraction from 4-5 min was collected. c, The purity of the final products was validated by 15% denaturing PAGE.

FIG. 6: Synergy of HD22-7A-DAB. The thrombin inhibition activity of HD22-7A-DAB was compared to HD23-7A-DAB, and HD22-7A-NH₂-mediated inhibition where the aptamer or the small molecule inhibitor was replaced with an inactive moiety, respectively. HD22 and DAB were used as controls in the assay.

FIG. 7: Simulation of enzyme inhibition by EXACT inhibitors with different K_{EA,I} based on the proposed two-step binding model. a. Simulations of enzyme inhibition were conducted using the rapid equilibrium assumption with the additional assumptions that K_{E,A}=1 nM, K_{E,I}=50 nM, α₁=1, α₂=0, α₃=0, and Eₜ=0.5 nM. Simulated inhibition curves are shown for the indicated values of K_{EA,I}. b. The maximum inhibition and the concentration of AI yielding 50% of the maximum inhibition (IC₅₀) were determined from the simulated curves, demonstrating that a lower K_{EA,I} resulted in lower IC₅₀ and higher inhibition max.

FIG. 8: Determination of binding kinetics and dissociation constants by bio-layer interferometry. a-d, Interactions of immobilized HD22-7A-DAB (a), HD22-7A-NH₂ (b), HD-23-7A-DAB (c), and HD23-7A-NH₂ (d) with different concentrations of thrombin. e, the fitted binding kinetics and dissociation constants of HD22-7A-DAB and derivatives.

FIG. 9: Determination of binding stoichiometry of HD22-7A-DAB to thrombin using size exclusion chromatography. a, the elution profiles of protein standard, HD22-7A-DAB (1.25 nmole), thrombin (1.25 nmole) and HD22-7A-DAB thrombin mixture (yellow: 1.25 nomle each). b, apparent molecular weight of HD22-7A-DAB, thrombin and HD22-7A-DAB thrombin complex. When HD22-7A-DAB complexes with thrombin, the total surface area exposed to the solvent is reduced. Thus it appears to behave as a smaller molecule in the column, which leads to an apparent molecular weight (50 KDa) that's lower than the sum of its components (70 KDa).

FIG. 10: K_{EA,I} of EXACT inhibitor with different linker lengths calculated by global fitting of the data in FIG. 1f.

FIG. 11: Factor Xa inhibition by 11F7t-20A-DAB and other inhibitors in a fluorogenic substrate cleavage assay. 11F7t-20A-DAB (yellow) showed a significantly higher inhibitory effect of thrombin than DAB, 11F7t, or their equimolar mixture, and 11F7t-20A-DAB can be efficiently reversed by addition of an antidote oligonucleotide (A05.4) (green).

FIG. 12: Potency of factor Xa EXACT inhibitors containing different active site inhibitors. a, chemical structure of DAB and APX. b, Factor Xa inhibition by free active site inhibitors (APX and DAB) and EXACT inhibitors (11F7t-20A-APX and 11F7t-20A-DAB) in a fluorogenic substrate cleavage assay.

FIG. 13: Comparison of different antidotes for HD22-7A DAB. Thrombin activity (%) of each sample is measured by fluorogenic activity assay and normalized with the thrombin activity in the absence of inhibitor as 100%. Samples AO1-5 show thrombin activity in the presence of 250 nM HD22-7A-DAB and 2 μM of different AOs. Sample No AO and DAB ctrl show thrombin activity in the presence of 250 nM HD22-7A-DAB and DAB, respectively. One-way ANOVA test was used to compare between two sets of data. *, P<0.05, ns, not significant.

FIG. 14: Anticoagulation activity of EXACT inhibitor HD22-7A-DAB (red; open shapes) in comparison with UFH (black; filled shapes). Different concentrations HD22-7A-DAB or UFH were added to normal human plasma and incubated for 5 min at 37° C. and a, Thrombin time (TT), b, Prothrombin time (PT), and c, Partial thromboplastin time (aPTT) assays were performed to characterize anticoagulant efficacy. To characterize antidote reversal of HD22-7A-DAB, another 5-min incubation of antidote oligo (2 μM) with the plasma-HD22-7A-DAB mixture was performed. In the TT assay, plasma did not clot within the measurable range of the coagulation analyzer (999 sec) when the concentration of HD22-7A-DAB exceeds 16 nM.

FIG. 15. The potency of 11F7t-APX conjugates with different linker lengths and chemistry a, 2′OMeA; b, abasic site; c, PEG3 were tested in the fluorogenic substrate cleavage assay.

FIG. 16. End modification of aptamer for higher plasma stability. (a) 3′ modification of HD22-DAB conjugate with invdT or cholesterol modification retain high inhibition potency to thrombin in fluorogenic substrate assay. (b) End modification of HD22-7A-DAB with 3′ invdT or cholesterol prolong high life in plasma. Plasma was prepared from whole blood collected from healthy volunteers that was supplemented with 10 U/mL of heparin to prevent coagulation. 500 nM of aptamer-drug conjugate were incubated with 200 μL of the plasma at 37° C. 20 μL of plasma reaction was collected at different time point (0, 10, 30, 60, 120, 360 min) and immediately vortexed with 100 μL phosphate buffer saline, 100 μL chloroform, and 200 μL methanol to quench nucleases. A mixture of 100 μL water and 100 μL chloroform was further added to extract the nucleic acids. The mixture was vortexed and centrifuged 500 rcf×20 min, and the aqueous phase containing nucleic acids was collected, lyophilized, reconstituted in water, and analyzed on a 15% polyacrylamide gel containing 7M urea.

FIG. 17. Thrombin inhibition by Tog25-DAB conjugates in fluorogenic substrate assay. (a) Tog25-20A-DAB showed significantly higher inhibitory effect to thrombin compared with DAB, Tog25-20A-NH₂, or their equimolar mixture, and can be efficiently reversed by an antisense oligo. (b) The potency Tog25-DAB conjugates with different linker lengths were tested. (c) The potency Tog25-DAB conjugates with different linker positions were tested. (d) The potency Tog25-DAB conjugates with different linker chemistry were tested.

FIG. 18. Thrombin inhibition by HD1-DAB conjugates in fluorogenic substrate assay. (a) HD1-12A-DAB showed significantly higher inhibitory effect to thrombin compared with DAB, HD1, or their equimolar mixture, and can be efficiently reversed by an antisense oligo. (b) The potency HD1-DAB conjugates with different linker positions were tested. (c) The potency HD22-DAB conjugates with different linker lengths were tested.

FIG. 19. Various linker chemistry used in this work.

FIG. 20: Thrombin inhibition by HD22 3′ DAB conjugates in fluorogenic substrate assay. (a) The potency HD22 3′ DAB conjugates with different linker lengths (1 to 3 nt) were tested, and HD22-7A-DAB was used as a control. (b) HD22-2T-DAB's potency was tested in the presence and absence of 2 μM antidote strand (3TAO1). HD22-7A-DAB in the presence and absence of AO2 was used as control.

FIG. 21: Anticoagulation activity of 11f7t-20A-APX. Different concentrations of 11f7t-20A-NH₂, APX, equimolar mixture of 11f7t-20A-NH₂ and APX, and 11f7t-20A-APX were added to normal human plasma and incubated for 5 min at 37° C. and aPTT assays were performed to characterize their anticoagulation activity.

### DETAILED DESCRIPTION OF THE INVENTION

Potent and selective inhibition of the structurally homologous proteases of coagulation poses challenges for drug development. Disclosed herein are aptamer-direct inhibitor conjugates comprising a nucleic acid aptamer binding to the exosite (EXosite) of a protease and a small molecule inhibitor binding to the catalytic active (ACTive) site of the same protease (called EXACT inhibitors herein). The resulting conjugates are capable of simultaneously inhibiting exosite and catalytic sites of the protease with potency far exceeding the constructing aptamer or small molecule inhibitor alone or their equimolar mixture. The aptamer component within the EXACT inhibitor not only enhances the potency of the small-molecule active site inhibitor by hundreds of folds but also dictates protease specificity and enables the reversal of inhibition by an antidote that disrupts aptamer binding. Interestingly, the exosite binding aptamer can also increase affinity for an otherwise weak binding of a small molecule inhibitor. These aptamer-inhibitor conjugates can be coupled via a linker which can vary in length and composition. Thus, described herein is a generalizable strategy for the generation of selective, portent and rapidly reversible EXACT inhibitors which can be rapidly created against many enzymes through simple oligonucleotide conjugation for numerous research and therapeutic applications.

### Compositions

Some embodiments of the present disclosure provide a composition for inhibiting a protease or coagulation target comprising a nucleic acid aptamer covalently linked to a small molecule inhibitor, wherein the nucleic acid aptamer targets the exosite of the proteases or the coagulation target and wherein the small molecule inhibitor inhibits the active site of the protease or the coagulation target.

Some embodiments of the present disclosure provide a composition for inhibiting a protease. A protease is an enzyme that catalyzes proteolysis, breaking down proteins into smaller polypeptides or single amino acids, and spurring the formation of new protein products. They do this by cleaving the peptide bonds within proteins by hydrolysis. Examples of proteases include, but are not limited to thrombin, coagulation Factors IIa, VIIa, IXa, Xa, XIa and XIIa. The exosite of a protease is a secondary binding site, different from the active site. The active site is the region of an enzyme where substrate molecules bind and undergo a chemical reaction.

Some embodiments of the present disclosure provide a composition for inhibiting a coagulation target. Coagulation, also known as clotting, is the process by which blood changes from a liquid to a gel, forming a blood clot. It potentially results in hemostasis, the cessation of blood loss from a damaged vessel, followed by repair. Coagulation targets are those proteins or nucleic acids that participate in the process of coagulation. Coagulation targets may also be called clotting factors or coagulation factors and may be classified into three groups including fibrinogen family, vitamin K dependent proteins and contact family proteins. Coagulation factors include clotting factor numbers I through XX.

Some embodiments of the present disclosure provide a composition for inhibiting a protease or coagulation target comprising a nucleic acid aptamer. As used herein, the term “aptamer” or “nucleic acid aptamer” refers to single-stranded oligonucleotides that bind specifically to targets molecules with high affinity. Target molecules may include, without limitation, proteins, lipids, carbohydrates, or other types of molecules. Aptamers can be generated against target molecules, such as soluble coagulation proteins, by screening combinatorial oligonucleotide libraries for high affinity binding to the target (See, e.g., Ellington and Szostak, Nature 1990; 346: 8 18-22 (1990), Tuerk and Gold, Science 249:505-10 (1990)). The aptamers disclosed herein may be synthesized using methods well-known in the art. For example, the disclosed aptamers may be synthesized using standard oligonucleotide synthesis technology employed by various commercial vendors including Integrated DNA Technologies, Inc. (IDT), Sigma-Aldrich, Life Technologies, or Bio-Synthesis, Inc. Aptamers may be constructed from naturally occurring or non-naturally occurring nucleic acids and/or amino acids. Aptamers may comprise modified nucleotides, for example modifications which increase the stability of the nucleic acid. Examples of nucleic acid modifications include NH₂ amine modification, (2′OMeA) 2′-O-methyl modification, (2′FC) 2′ Fluorine C modification, (2′FU) 2′ Fluorine U modification and invdT inverted T modifications. In some embodiments, aptamers comprise one or more detectable labels or small molecule inhibitor. Aptamers of the present disclosure include those which bind to coagulation targets, including, but not limited to, VWF, FVIIa, FIXa, FXa, prothrombin, and thrombin. Examples of aptamers include, but are not limited to, HD1, HD22, Tog25t, 11f7t, R9D-14T, 11.16, 12.7, 7S-1, 9.3T, 17-1, 7S-2, 7K-2, 7K-3, 7K-58, 7K-5, 9D-24, 10S-20, 9D-6, 9D-10, 9D15, 9D20, 9D-31 (See also, U.S. Pat. Nos. 8,367,627; 9,687,529; 10,660,973; U.S. Pat. Pub. No. 2020/0353102; and Thromb Res. 2017 156:134-141).

Aptamers can be selected by in vitro screening of complex nucleic-acid based combinatorial shape libraries (>1014 shapes per library) employing a process termed SELEX (for Systematic Evolution of Ligands by EXponential Enrichment) (Tuerk et al, Science 249:505-10 (1990)). The SELEX process consists of iterative rounds of affinity purification and amplification of oligonucleotides from combinatorial libraries to yield high affinity and high specificity ligands. Combinatorial libraries employed in SELEX can be front-loaded with 2′modified RNA nucleotides (e.g., 2′fluoro-pyrimidines) such that the aptamers generated are highly resistant to nuclease-mediated degradation and amenable to immediate activity screening in cell culture or bodily fluids. (See also U.S. Pat. Nos. 5,670,637, 5,696,249, 5,843,653, 6,110,900, 5,686,242, 5,475,096, 5,270,163 and WO 91/19813.) The aptamers presented herein are generally RNA aptamers or modified RNAs comprising modifications to increase the stability of the RNAs, such as phosphothiorate backbones, sugar modifications, s′ cap structures or inverted dT at the 3′ end to render the RNA aptamer less susceptible to RNases.

In some embodiments the HD22 aptamer is used (SEQ ID NO: 1). HD22 is thrombin binding aptamer. HD22 recognizes the thrombin exosite II. The nucleotides G23, T24, G25, A26, C27 of the HD22 double-stranded portion and the nucleotides T9, T18, T19, G20 of the G4 portion facilitate interaction with exosite II of thrombin. Since thrombin external site II is a positively charged motif, it creates many ion pairs with the HD22 backbone, especially in the duplex region. Hydrophobic interactions were observed in the G4 region (T9, T18 and T10), which stabilized complex formation. Furthermore, the interaction with thrombin improved the thermal stability of the HD22 structure and resulted in an increase in the melting temperature from 36° C. to 48° C.

In some embodiments, a Xa/FXa aptamer designated 11F7t is used (SEQ ID NO: 9). 11f7t is a 37-base RNA oligonucleotide (Buddai et al, J. Biol. Chem. 285:5212-5223 (2010)). The base sequence of that aptamer includes multiple 2′-fluoropyrimidines, and its 3′ terminal base is an inverted deoxythymidine. 11f7t inhibits thrombin formation catalyzed by prothrombinase, the complex of FXa, factor Va (FVa), and calcium ions assembled on a phospholipid surface, by inhibiting the interaction between FXa and FVa (Buddai et al, J. Biol. Chem. 285:5212-5223 (2010)). 11f7t binds FX as well as FXa (Buddai et al, J. Biol. Chem. 285:5212-5223 (2010)).

In some embodiments the Tog25 RNA aptamer is used (SEQ ID NO: 12). Tog25t is a thrombin-targeting aptamer (Long, S. B., et al. RNA. 14, 2504-2512 (2008). Tog25t binds to human thrombin and produces a modest extension in aPTT clotting time at relatively high concentrations of approximately 1 μM. Tog25 binds to exosite II of thrombin, thus inhibiting thrombin-mediated platelet activation and has a traditional stem-loop structure with an internal bulge.

In some embodiments, the HD1 aptamer is used (SEQ ID NO: 15). HD1 binds exosite 1 on thrombin and blocks its clotting activity. HD1 also binds prothrombin and inhibits its activation by prothrombinase (Kretz et al. JBC v.281(49)2006).

Some embodiments of the present disclosure provide a composition for inhibiting a protease or coagulation target comprising a nucleic acid aptamer linked to a small molecule inhibitor. A small molecule inhibitor is a compound with a low molecular weight that can inhibit any portion of a molecule. Examples of small molecule inhibitors as used herein include, but are not limited to, dabigatran, argatroban, apixaban, edoxaban, razaxaban, rivaroxaban, otamixaban, DPC-423, DCP-602, SSR-182289, LB-30057, asundexian, or benzamidine.

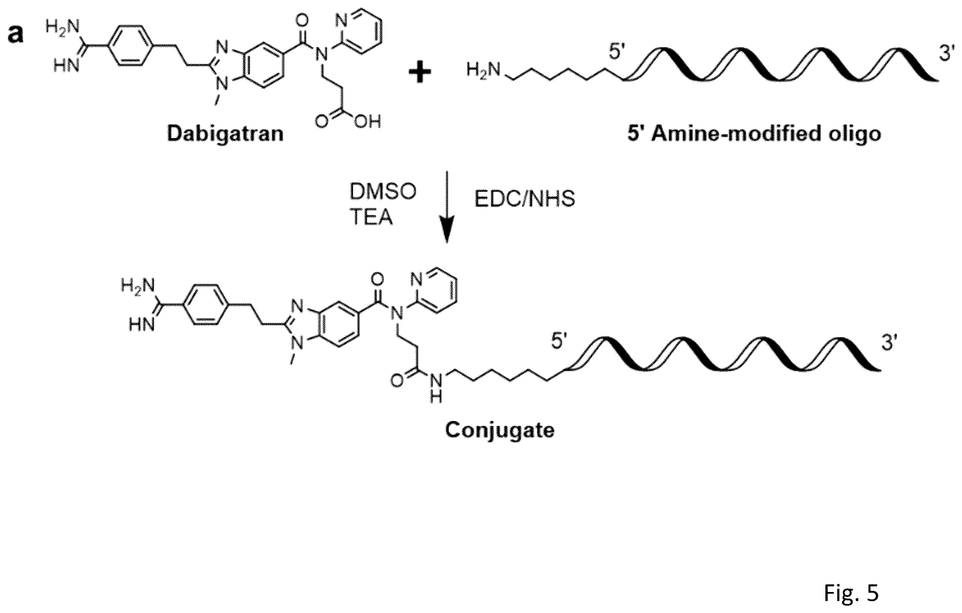

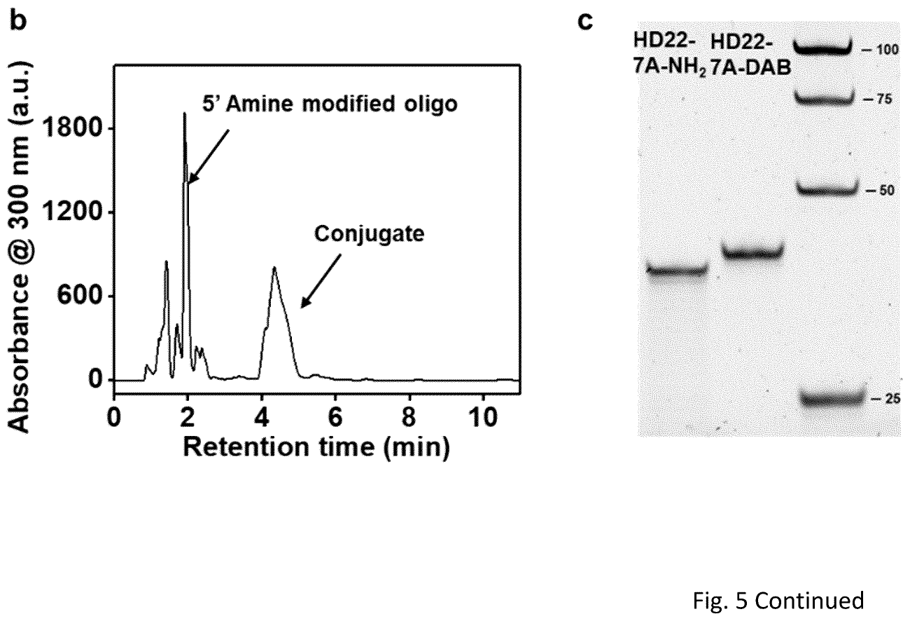

The aptamer and small molecule inhibitor may be covalently connected directly to each other or may be connected via a linker or spacer. The linker may be made of any natural or non-natural nucleic acid and may be at least one nucleotide in length. The linker may optionally comprise more than one nucleotide, including 2, 3, 4, 5, 6, 7, 8, 9, 10, 11, 12, 13, 14, 15, 20, 25, 30, 35, 40 or more, or any value in-between in length. For example, the linker may be made of adenosine, thymidine or uracil. The linker may connect or be covalently bonded to the 5′ or 3′ end of the aptamer. Means of coupling a nucleic acid aptamer, linker and small molecule are known in the art. Such examples include 1-Ethyl-3-(3-dimethylaminopropyl) carbodiimide (EDC) alone or in combination with N-hydroxysuccinimide (NHS) or sulfoNHS, (N,N′-dicyclohexane carbodiimide) (DCC) or 1,1′-Carbonyldiimidazole (CDI). One example of potential linkage chemistry is shown in FIG. 5. The nucleic acid linker may also comprise a modified nucleic acid. Modifications may comprise those which increase stability of the nucleic acid. Nucleic acid modifications are known in the art and include, but are not limited to 2′OMe, 2′Fluoro, and 2′-MOE. For example, the linker may comprise 2′-OMe adenosine (2′-OMeA). In some embodiments, the linker may comprise a moiety that is not a nucleotide. Examples of non-nucleic acid linkers include Abasic site (polydeoxyribose), polyethylene glycol oligomers including PEG3 or other inert polymer linkers. As shown throughout Example 1, a linker can span than the distance or be between the active site and exosite or be longer than the distance between the two. Aptamers with various linker lengths, chemistries, and position relative to the aptamer are included in Table 1.

Compositions described herein include the HD22 aptamer linked via a 0, 2, 5, 7, 10, 15, 20, 25 or 30 adenosine or 2′OMeA linker to dabigatran. Compositions described herein also include HD22 linked via a 1, 2, or 3 thymidine linker to dabigatran. Compositions described herein include the 11 f7t aptamer linked via a 20 adenosine linker to dabigatran, or with a 3, 5, 8, 13, 20 adenosine or 2′O-MeA linker to apixaban. Compositions described herein also include the 11 f7t aptamer linked via 3, 5, 8, 13, or 20 Abasic site to apixaban, or via 1, 2, 3, 5, or 8 PEG3 to apixaban. Compositions further include Tog25 linked via a 35, 30, 20, or 10 adenosines or 2′OMeA to dabigatran. Compositions also include the HD1 aptamer linked via 20, 12, 9, 7, 5 or 0 adenosine to dabigatran. These linkers may be attached 5′ or 3′ to the aptamers.

Some embodiments of the present disclosure provide a composition wherein the composition is more potent than the aptamer or small molecular inhibitor alone or their equimolar mixture. As used herein, a composition is more potent if the effect of the composition is greater than, faster than or can be used to the same effect at a lower concentration than the alternative. The aptamer-inhibitor compositions described herein may also increases affinity of an otherwise weak binding small molecule inhibitor.

### Methods

Methods for inhibiting coagulation are also provided herein. Methods for inhibiting coagulation include contacting a site of coagulation or potential coagulation with any of the compositions described herein. These methods may include administering the composition to a subject in need. Methods of using the composition described herein may also include use wherein the composition is not administered directly to a subject, but rather used in conjunction with a procedure or machine for example, hemodialysis or extracorporeal membrane oxygenation (ECMO).

The term “subject” refers to both human and non-human animals. The term “non-human animals” includes all vertebrates, e.g., mammals and non-mammals, such as non-human primates, sheep, dog, cat, horse, cow, mice, chicken, amphibians, reptiles and the like. In some embodiments, the subject is a human patient.

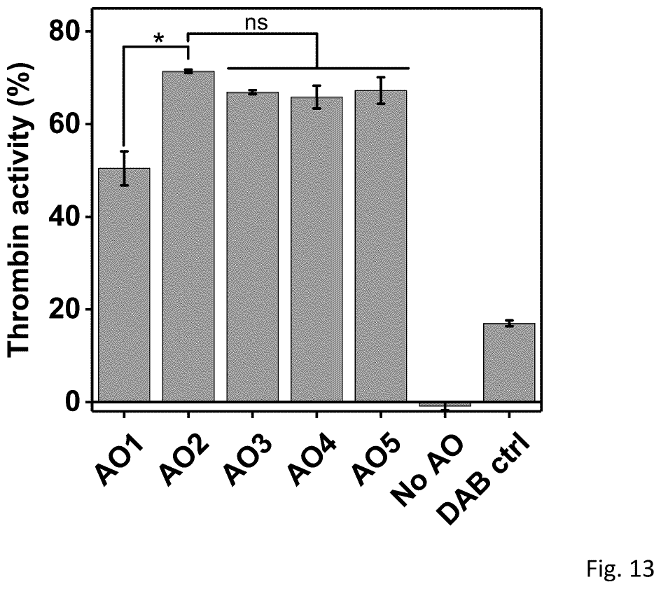

The method may further comprise administering an antidote to a subject, wherein the antidote inhibits the binding of the composition to its target. An antidote is a complementary oligonucleotide, the base sequence of which is complementary to all or part of an aptamer's base sequence and/or linker and can neutralize an aptamer's anticoagulant effect or the anticoagulation effect of the aptamer-small molecule inhibitor composition. In particular, the complementary antidote oligonucleotides (AO) can disrupt the structure of the aptamer or the linker. Several polyamines, including protamine, can effect non-specific neutralization of aptamers. Antidotes also include pharmaceutically acceptable members of a group of positively charged compounds, including lipids, and natural and synthetic polymers that can bind nucleic acid molecules. Examples include of PPA-DPA, CDP, CDP-Im, PAMAM, and HDMB. (See also U.S. application Ser. No. 12/588,016, U.S. Pat. No. 9,340,591B2, Dyke, Circulation 114(23):2490-7 (2006), Rusconi et al, Nat Biotechnol. 22(11):1423-8 (2004), Rusconi et al, Nature 419(6902):90-4 (2002)); Published U.S. Application No. 20030083294). Joachimi et al (J. Am. Chem. Soc. 129:3036-3037 (2007))). Antidotes of the present disclosure may bind to the linker and part of or all of the aptamer. Antidotes of the present disclosure include AO2 (SEQ ID NO: 4) and 3TAO1 (SEQ ID NO: 8), Tog25-AO (SEQ ID NO: 13) and HD1-AO (SEQ ID NO: 17). Antidotes that are complementary oligonucleotides antidotes generally need to be capable of binding to at least 7 nucleotides of the aptamer and/or nucleotide-based linker molecule. Thus oligonucleotide-based antidotes may be reverse complementary to at least 7, 8, 10, 12, 14, 15, 16, 17, 18, 19, 20 or more nucleotides of the aptamer and/or linker. The antidote can be as long as the aptamer-linker or shorter than the full-length aptamer-linker. The antidote need not be 100% reverse complementary to the aptamer-linker, but longer antidotes will be needed if the antidotes is less than 100% reverse complementary to the aptamer-linker. For those compositions comprising a nucleotide linker the antidote may be more effective if it includes at least a portion reverse complementary to the linker. See FIG. 13 and the Examples.

Methods of treating a subject in need of anti-coagulation therapy are also provided herein. These methods comprise, administering a composition described herein to the subject for a period of time sufficient to allow a reduction in coagulation in the subject and may further comprise administering an oligonucleotide antidote, wherein the oligonucleotide antidote reverses the anti-coagulation activity of the composition. Antidotes may be complementary antidote oligonucleotides (AO) that disrupt the folded structure of the aptamer or complementary oligonucleotides that bind to the linker and disrupt binding of the aptamer and/or the small molecule inhibitor to the exosite and active site of the protease or coagulation pathway member.

Methods for inhibiting coagulation or for treating subject in need of anti-coagulation therapy include administering compositions described herein. As used herein, the term “administering” a composition, such as a nucleic acid aptamer linked to a small molecule inhibitor to a subject, animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

Methods for inhibiting coagulation or for treating subject in need of anti-coagulation therapy include method for preventing or treating thrombosis, cardiopulmonary bypass surgery, percutaneous coronary intervention, stroke, deep vein thrombosis or surgery or subjects who are suffer from or are undergoing treatment for the same.

Methods for increasing the affinity of a protease to a target are also contemplated. For example, dabigatran is a selective thrombin active site inhibitor, but may bind to other proteases such as Xa with weaker affinity. As described herein, the use of 11F7t, which is a Xa directed aptamer conjugated via a linker to dabigatran increases the affinity of dabigatran for Xa.

It will also be appreciated that the specific dosage of composition and antidotes administered in any given case will be adjusted in accordance with the composition or compositions being administered, the suspected abnormality to be treated, the condition of the subject, and other relevant medical factors that may modify the activity of the composition or the response of the subject, as is well known by those skilled in the art. For example, the specific dose for a particular subject depends on age, body weight, general state of health, diet, the timing and mode of administration, the rate of excretion, medicaments used in combination as well as other factors. Dosages for a given patient can be determined using conventional considerations.

The maximal dosage for a subject is the highest dosage that does not cause undesirable or intolerable side effects. The number of variables in regard to an individual treatment regimen is large, and a considerable range of doses is expected. The route of administration will also impact the dosage requirements.

The effective dosage amounts described herein refer to total amounts administered, that is, if more than one composition is administered, the effective dosage amounts correspond to the total amount administered. The compositions described herein can be administered as a single dose or as divided doses. For example, the composition may be administered two or more times separated by 30 minutes, 1 hour, 2 hours, 4 hours, 6 hours, 8 hours, 12 hours, a day, two days, three days or more.

The compositions and antidotes described herein may be administered one time or more than one time to the subject to effectively treat the subject. Precise amounts of effective composition and antidotes required to be administered depends on the judgment of the practitioner and may be peculiar to each subject or condition being treated. The compositions and antidotes described herein may also be prepared for administration as pharmaceutical preparations and may include a pharmaceutically acceptable carrier or excipient. Examples of pharmaceutically acceptable carriers suitable for use include, but are not limited to, water, buffered solutions, glucose solutions, oil-based or bacterial culture fluids. Additional components of the compositions may suitably include, for example, excipients such as stabilizers, preservatives, diluents, emulsifiers and lubricants. Examples of pharmaceutically acceptable diluents include stabilizers such as carbohydrates (e.g., sorbitol, mannitol, starch, sucrose, glucose, dextran), proteins such as albumin or casein, protein-containing agents such as bovine serum or skimmed milk and buffers (e.g., phosphate buffer).

### Additional Definitions

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

This application includes methods of treating a human or animal by administration of compositions described herein. These methods of treatment or administration may also be reformulated as compositions for use in medical treatments, second medical use or other means of covering the compositions or their use.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1—Aptameric Hirudins: Synthetic Potent, Selective, and Reversible EXosite-ACTive Site (EXACT) Inhibitors

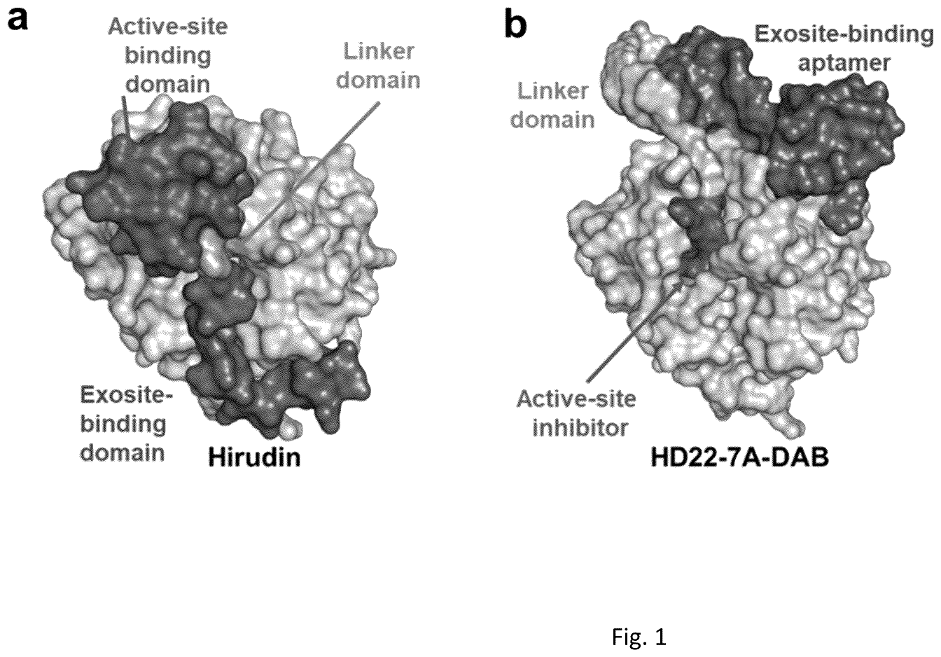

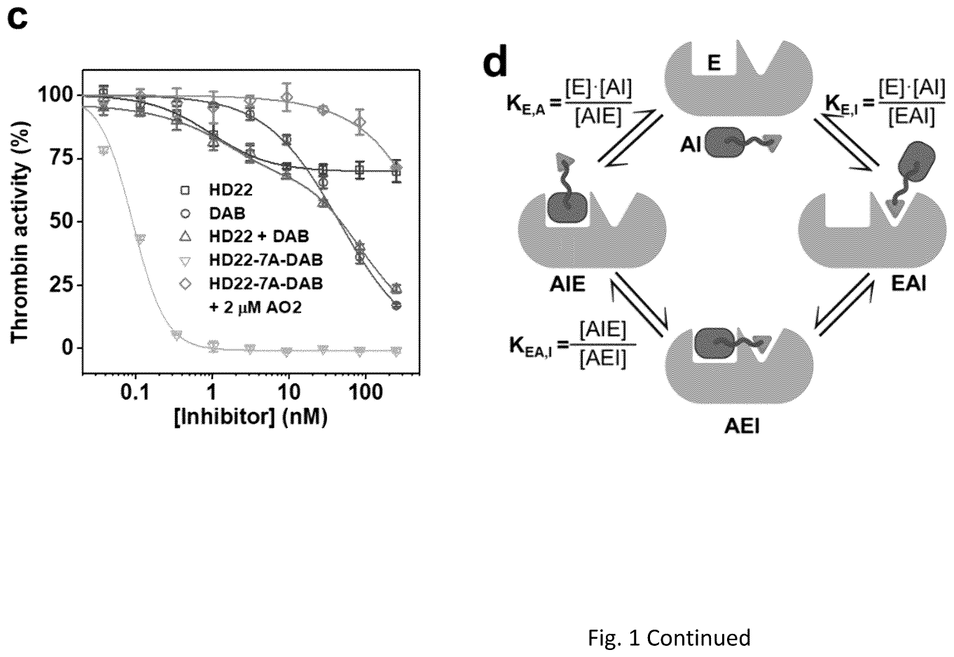

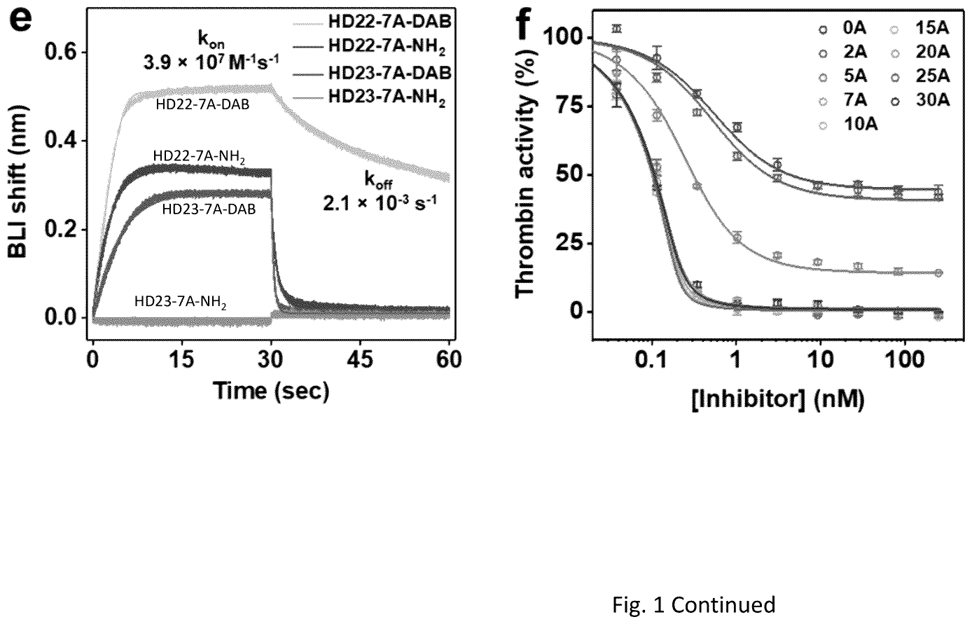

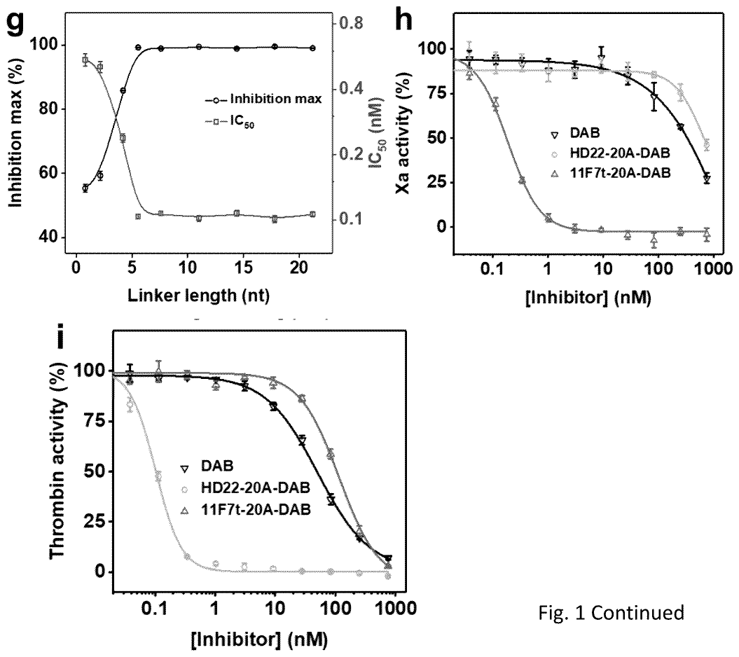

Hematophagous organisms, such as leeches, ticks, mosquitos and nematodes, use potent inhibitors of the coagulation proteases to acquire blood meals.¹ These inhibitors are frequently peptides that engage the target protease through exosite and active site interactions to fulfill the biological needs of high potency and rapid onset of action during feeding.²⁻⁹ This strategy is exemplified by hirudin from the medicinal leech that targets thrombin using an N-terminal active site binding motif linked to a C-terminal region that binds anion binding exosite I (ABE1) (FIG. 1a).² The two motifs contribute synergistically to binding and achieve a 10⁻¹¹M inhibition constant (Kᵢ) which is greatly affected when binding by either domain is impaired.^{10,11} Tick anticoagulant peptide (TAP) secreted by the Ornithodoros moubata⁷ also achieves potent inhibition of factor Xa by simultaneously binding the active site and an extended surface on the protease, and inhibits free FXa (Kᵢ=0.18 nM) and complexed FXa within prothrombinase (Kᵢ=5.3 μM) to achieve potent anticoagulation through an analogous mechanism.^{9,12}

The evolution of such bivalent inhibitors by different blood-feeding organisms inspired the inventors to pursue a synthetic but analogous bifunctional strategy using a small molecule active site inhibitor tethered to an exosite-binding aptamer. By evaluating the binding and inhibitory mechanisms of such EXosite and ACTive site (EXACT) inhibitors, the inventors found that the exosite-binding aptamer can be utilized to modulate the apparent binding affinity and target selectivity of the small molecule moiety. This feature can be used to manipulate the potency, specificity, and antidote-mediated reversibility of the EXACT inhibitors. This bioinspired EXACT inhibitor strategy is generalizable to different aptamer-small molecule combinations and thus represents a molecular engineering approach for generating potent, selective, and reversible inhibitors for a wide range of enzymes that are vital for health and dysregulated in disease.

Rational design of thrombin-binding EXACT inhibitor. The inventors investigated whether an EXACT inhibitor against thrombin could be rationally created de novo by conjugating a direct thrombin inhibitor dabigatran (DAB, Kᵢ=4.5 nM)¹³ to the DNA aptamer HD22¹⁴ (Table 1 and FIG. 5). HD22 binds to anion binding exosite 2 (ABE2) of thrombin with high affinity (K_{D}=0.5 nM) and high selectivity, but has weak anticoagulant activity as it does not bind either ABE1—the fibrinogen binding site or the active site.¹⁴ The crystal structure of the HD22-thrombin complex indicated that the distance between HD22's 5′ terminus and thrombin's active site is approximately 40 Å.¹⁵ Therefore, the inventors extended HD22's 5′ terminus with a poly-(7)-deoxyadenosine linker (estimated length of 49 Å)¹⁶ to facilitate the simultaneous binding of both the HD22 and the DAB moieties (FIG. 1b and Table 1). DAB's carboxyl group which is not directly involved in binding thrombin¹⁵ was then conjugated to the 5′ amino modifier at the terminal of the linker. The resulting EXACT inhibitor is named HD22-7A-DAB.

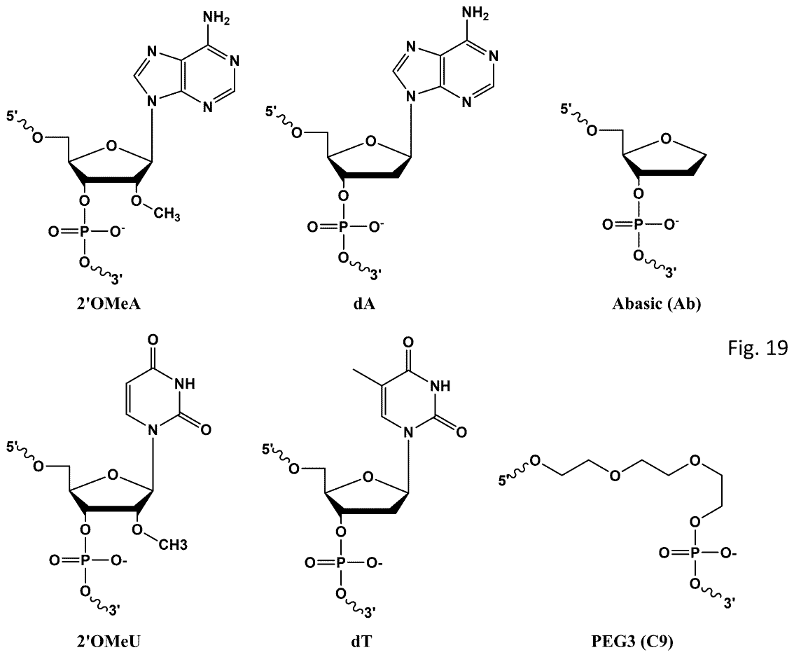

TABLE 1
Sequences used in this disclosure. The linker chemistries used in the work are shown
in FIG. 19. Linker or aptamer nucleotides may be modified, modification may comprise,
for example: Ribo-nucleotide, 2′-Fluoro modified nucleotide and 3′ Inverted dT.

| | | SEQ ID | Linker | Linker |
|---|---|---|---|---|
| Oligo Name | Sequence (5′→3′) | NO: | Chemistry | Position |
| HD22 | AGTCCGTGGTAGGGCAGGTTGGGGT | 1 | | |
| | GACT | | | |
| HD22-7A | AAAAAAAAGTCCGTGGTAGGGCAGG | 2 | dA | 5′ |
| | TTGGGGTGACT | | | |
| HD22-0A-DAB | DAB-C₆H₁₂- | 1 | | |
| | AGTCCGTGGTAGGGCAGGTTGGGGT | | | |
| | GACT | | | |
| HD22-2A-DAB | DAB-C₆H₁₂- | 1 | dA | 5′ |
| | A(2)AGTCCGTGGTAGGGCAGGTTGG | | | |
| | GGTGACT | | | |
| HD22-5A-DAB | DAB-C₆H₁₂- | 1 | dA | 5′ |
| | A(5)AGTCCGTGGTAGGGCAGGTTGG | | | |
| | GGTGACT | | | |
| HD22-7A-DAB | DAB-C₆H₁₂- | 1 | dA | 5′ |
| | A(7)AGTCCGTGGTAGGGCAGGTTGG | | | |
| | GGTGACT | | | |
| HD22-7A-DAB- | DAB-C₆H₁₂- | 1 | dA | 5′ |
| 3′CH | A(7)AGTCCGTGGTAGGGCAGGTTGG | | | |
| | GGTGACT-CH | | | |
| HD22-7A-DAB- | DAB-C₆H₁₂- | 1 | dA | 5′ |
| 3′invT | A(7)AGTCCGTGGTAGGGCAGGTTGG | | | |
| | GGTGACT-invdT | | | |
| HD22-7A-NH₂ | NH2-C₆H₁₂- | 1 | dA | 5′ |
| | A(7)AGTCCGTGGTAGGGCAGGTTGG | | | |
| | GGTGACT | | | |
| HD23-7A-DAB | DAB-C₆H₁₂- | 3 | dA | 5′ |
| | A(7)AGTCCGTAATAAAGCAGGTTAA | | | |
| | AATGACT | | | |
| HD22-10A-DAB | DAB-C₆H₁₂- | 1 | dA | 5′ |
| | A(10)AGTCCGTGGTAGGGCAGGTTGG | | | |
| | GGTGACT | | | |
| HD22-15A-DAB | DAB-C₆H₁₂- | 1 | dA | 5′ |
| | A(15)AGTCCGTGGTAGGGCAGGTTGG | | | |
| | GGTGACT | | | |
| HD22-20A-DAB | DAB-C₆H₁₂- | 1 | dA | 5′ |
| | A(20)AGTCCGTGGTAGGGCAGGTTGG | | | |
| | GGTGACT | | | |
| HD22-25A-DAB | DAB-C₆H₁₂- | 1 | dA | 5′ |
| | A(25)AGTCCGTGGTAGGGCAGGTTGG | | | |
| | GGTGACT | | | |
| HD22-30A-DAB | DAB-C₆H₁₂- | 1 | dA | 5′ |
| | A(30)AGTCCGTGGTAGGGCAGGTTGG | | | |
| | GGTGACT | | | |
| AO2 | AGTCACCCCAACCTGCCCTACCACG | 4 | | |
| | GACTTTTTTTT | | | |
| HD22-1T-DAB | AGTCCGTGGTAGGGCAGGTTGGGGT | 5 | dT | 3′ |
| | GACTT-C₆H₁₂O₂-DAB | | | |
| HD22-2T-DAB | AGTCCGTGGTAGGGCAGGTTGGGGT | 6 | dT | 3′ |
| | GACTTT-C₆H₁₂O₂-DAB | | | |
| CH-HD22-2T- | CH- | 6 | dT | 3′ |
| DAB | AGTCCGTGGTAGGGCAGGTTGGGGT | | | |
| | GACTTT-C₆H₁₂O₂-DAB | | | |
| HD22-3T-DAB | AGTCCGTGGTAGGGCAGGTTGGGGT | 7 | dT | 3′ |
| | GACTTTT-C₆H₁₂O₂-DAB | | | |
| 3TAO1 | AAAAAGTCACCCCAACCTGCCCTAC | 8 | | |
| | CACGGACT | | | |
| 11F7t | GAGAGCCCCAGCGAGAUAAUACUU | 9 | | |
| | GGCCCCGCUCUUT | | | |
| 11F7t-20A | AAAAAAAAAAAAAAAAAAAAGAGA | 10 | dA | 5′ |
| | GCCCCAGCGAGAUAAUACUUGGCCC | | | |
| | CGCUCUUT | | | |
| 11F7t-20A-DAB | DAB-C₆H₁₂- | 9 | 2′OMeA | 5′ |
| | (2′OMeA)(20)GAGAGCCCCAGCGAGA | | | |
| | UAAUACUUGGCCCCGCUCUUT | | | |
| 11F7t-20A-APX | APX-C₆H₁₂- | 9 | 2′OMeA | 5 |
| | (2′OMeA)(20)GAGAGCCCCAGCGAGA | | | |
| | UAAUACUUGGCCCCGCUCUUT | | | |
| 11F7t-20A-NH2 | NH₂-C₆H₁₂- | 9 | 2′OMeA | 5′ |
| | (2′OMeA)(20)GAGAGCCCCAGCGAGA | | | |
| | UAAUACUUGGCCCCGCUCUUT | | | |
| 11F7t-13A-APX | APX-C₆H₁₂- | 9 | 2′OMeA | 5′ |
| | (2′OMeA)(13)GAGAGCCCCAGCGAGA | | | |
| | UAAUACUUGGCCCCGCUCUUT | | | |
| 11F7t-8A-APX | APX-C₆H₁₂- | 9 | 2′OMeA | 5′ |
| | (2′OMeA)(8)GAGAGCCCCAGCGAGAU | | | |
| | AAUACUUGGCCCCGCUCUUT | | | |
| 11F7t-5A-APX | APX-C₆H₁₂- | 9 | 2′OMeA | 5′ |
| | (2′OMeA)(5)GAGAGCCCCAGCGAGAU | | | |
| | AAUACUUGGCCCCGCUCUUT | | | |
| 11F7t-3A-APX | APX-C₆H₁₂- | 9 | 2′OMeA | 5′ |
| | (2′OMeA)(3)GAGAGCCCCAGCGAGAU | | | |
| | AAUACUUGGCCCCGCUCUUT | | | |
| 11F7t-20Ab- | APX-C₆H₁₂- | 9 | Abasic | 5′ |
| APX | (Ab)(20)GAGAGCCCCAGCGAGAUAA | | | |
| | UACUUGGCCCCGCUCUUT | | | |
| 11F7t-13Ab- | APX-C₆H₁₂- | 9 | Abasic | 5′ |
| APX | (Ab)(13)GAGAGCCCCAGCGAGAUAA | | | |
| | UACUUGGCCCCGCUCUUT | | | |
| 11F7t-8Ab-APX | APX-C₆H₁₂- | 9 | Abasic | 5′ |
| | (Ab)(8)GAGAGCCCCAGCGAGAUAAU | | | |
| | ACUUGGCCCCGCUCUUT | | | |
| 11F7t-5Ab-APX | APX-C₆H₁₂- | 9 | Abasic | 5′ |
| | (Ab)(5)GAGAGCCCCAGCGAGAUAAU | | | |
| | ACUUGGCCCCGCUCUUT | | | |
| 11F7t-3Ab-APX | APX-C₆H₁₂- | 9 | Abasic | 5′ |
| | (Ab)(3)GAGAGCCCCAGCGAGAUAAU | | | |
| | ACUUGGCCCCGCUCUUT | | | |
| 11F7t-8C9-APX | APX-C₆H₁₂- | 9 | PEG3 | 5′ |
| | (C9)(8)GAGAGCCCCAGCGAGAUAAU | | | |
| | ACUUGGCCCCGCUCUUT | | | |
| 11F7t-5C9-APX | APX-C₆H₁₂- | 9 | PEG3 | 5′ |
| | (C9)(5)GAGAGCCCCAGCGAGAUAAU | | | |
| | ACUUGGCCCCGCUCUUT | | | |
| 11F7t-3C9-APX | APX-C₆H₁₂- | 9 | PEG3 | 5′ |
| | (C9)(3)GAGAGCCCCAGCGAGAUAAU | | | |
| | ACUUGGCCCCGCUCUUT | | | |
| 11F7t-2C9-APX | APX-C₆H₁₂- | 9 | PEG3 | 5′ |
| | (C9)(2)GAGAGCCCCAGCGAGAUAAU | | | |
| | ACUUGGCCCCGCUCUUT | | | |
| 11F7t-1C9-APX | APX-C₆H₁₂- | 9 | PEG3 | 5′ |
| | (C9)GAGAGCCCCAGCGAGAUAAUAC | | | |
| | UUGGCCCCGCUCUUT | | | |
| AO5.4 | TATTATCTCGCTGGGGCTCTCTTTTT | 11 | | |
| | TTTTTTTTTTTTTTT | | | |
| Tog25-20A-NH2 | NH2-C₆H₁₂- | 12 | 2′OMeA | 5′ |
| | (2′OMeA)(20)GGGAACAAAGCUGAAG | | | |
| | UACUUACCCT | | | |
| Tog25-AO | TTCTGCTTTGTTCCCTTTTTTTTTTTT | 13 | | |
| | TTTTTTTT | | | |
| Tog25-20A | AAAAAAAAAAAAAAAAAAAAGGGA | 14 | 2′OMeA | 5′ |
| | ACAAAGCUGAAGUACUUACCCT | | | |
| Tog25-35A- | DAB-C₆H₁₂-(2′OMeA)(35) | 12 | 2′OMeA | 5′ |
| DAB | GGGAACAAAGCUGAAGUACUUACC | | | |
| | CT | | | |
| Tog25-30A- | DAB-C₆H₁₂-(2′OMeA)(30) | 12 | 2′OMeA | 5′ |
| | GGGAACAAAGCUGAAGUACUUACC | | | |
| DAB | CT | | | |
| Tog25-20A- | DAB-C₆H₁₂-(2′OMeA)(20) | 12 | 2′OMeA | 5′ |
| | GGGAACAAAGCUGAAGUACUUACC | | | |
| DAB | CT | | | |
| Tog25-10A- | DAB-C₆H₁₂- 2′OMeA)(10) | 12 | 2′OMeA | 5′ |
| DAB | GGGAACAAAGCUGAAGUACUUACC | | | |
| | CT | | | |
| Tog25-0A-DAB | DAB-C₆H₁₂- | 12 | | |
| | GGGAACAAAGCUGAAGUACUUACC | | | |
| | CT | | | |
| Tog25-3′20A- | GGGAACAAAGCUGAAGUACUUACC | 12 | 2′OMeA | 3′ |
| DAB | CT(2′OMeA)(20)-C₆H₁₂O₂-DAB | | | |
| Tog25-30dA- | DAB-C₆H₁₂- | 12 | dA | 5′ |
| DAB | A(30)GGGAACAAAGCUGAAGUACUU | | | |
| | ACCCT | | | |
| Tog25-30U- | DAB-C₆H₁₂- | 12 | 2′OMeU | 5′ |
| DAB | (2′OMeU)(30)GGGAACAAAGCUGAAG | | | |
| | UACUUACCCT | | | |
| HD1 | GGTTGGTGTGGTTGG | 15 | | |
| HD1-20A | AAAAAAAAAAAAAAAAAAAAGGTT | 16 | dA | 5′ |
| | GGTGTGGTTGG | | | |
| HD1-AO | TTTTTTTTTTTTTTTTTTTTCCAACCA | 17 | | |
| | CACCAACC | | | |
| DAB-20A-HD1 | DAB-C₆H₁₂- | 15 | dA | 5′ |
| | A(20)GGTTGGTGTGGTTGG | | | |
| HD1-20A-DAB | GGTTGGTGTGGTTGG-A(20)-C₆H₁₂O₂- | 15 | dA | 3′ |
| | DAB | | | |
| HD1-12A-DAB | GGTTGGTGTGGTTGG-A(12)-C₆H₁₂O₂- | 15 | dA | 3′ |
| | DAB | | | |
| HD1-9A-DAB | GGTTGGTGTGGTTGG-A(9)-C₆H₁₂O₂- | 15 | dA | 3′ |
| | DAB | | | |
| HD1-7A-DAB | GGTTGGTGTGGTTGG-A(7)-C₆H₁₂O₂- | 15 | dA | 3′ |
| | DAB | | | |
| HD1-5A-DAB | GGTTGGTGTGGTTGG-A(5)-C₆H₁₂O₂- | 15 | dA | 3′ |
| | DAB | | | |
| HD1-0A-DAB | GGTTGGTGTGGTTGG-C6H1202-DAB | 15 | | |

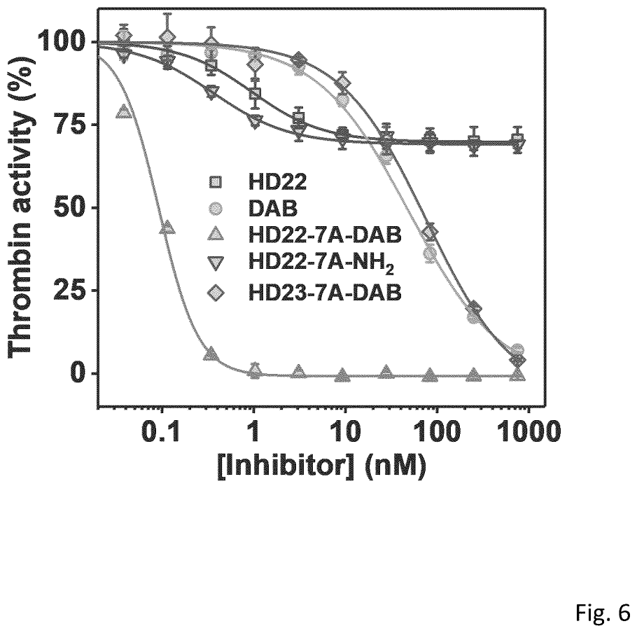

HD22-7A-DAB demonstrates extraordinary thrombin-binding affinity and inhibitory potency via synergistic binding. Initial velocity studies of fluorescent peptidyl substrate cleavage by thrombin were used to assess protease inhibition by the EXACT inhibitor (FIG. 1c). DAB alone inhibited thrombin in this assay with an IC₅₀ (the concentration of inhibitor yielding 50% of the maximum inhibition) of 50 nM, a value similar to that previously reported.¹⁷ On the other hand, exosite-binding HD22 does not directly block the active site and despite having a low IC₅₀ of 0.95 nM, it can only inhibit thrombin activity by 30% at maximum. Strikingly, HD22-7A-DAB inhibited thrombin with an IC₅₀ of 0.1 nM, indicating a potency 500-fold higher than DAB and the aptamer-small molecule conjugate completely inhibits thrombin activity at a concentration of 1 nM (FIG. 1c). The marked inhibitory potency of the EXACT inhibitor very likely results from the synergistic binding of thrombin by its HD22 and DAB moieties, since an equimolar mixture of free HD22 and free DAB achieved only the additive inhibitory effects of those two molecules (FIG. 1c). The inventors controlled for non-specific aptamer effects by generating a variant the EXACT inhibitor containing an aptamer with a point mutation that abrogates its high affinity binding to thrombin.¹⁸ This mutated EXACT inhibitor derivative, HD23-7A-DAB, has similar potency as DAB alone, confirming that exosite binding by the aptamer is required for potent thrombin inhibition by the parental EXACT inhibitor (FIG. 6). Similarly, replacing the DAB moiety of HD22-7A-DAB with a non-binding amine group (HD22-7A-NH₂) also yielded a derivative that did not significantly inhibit thrombin.

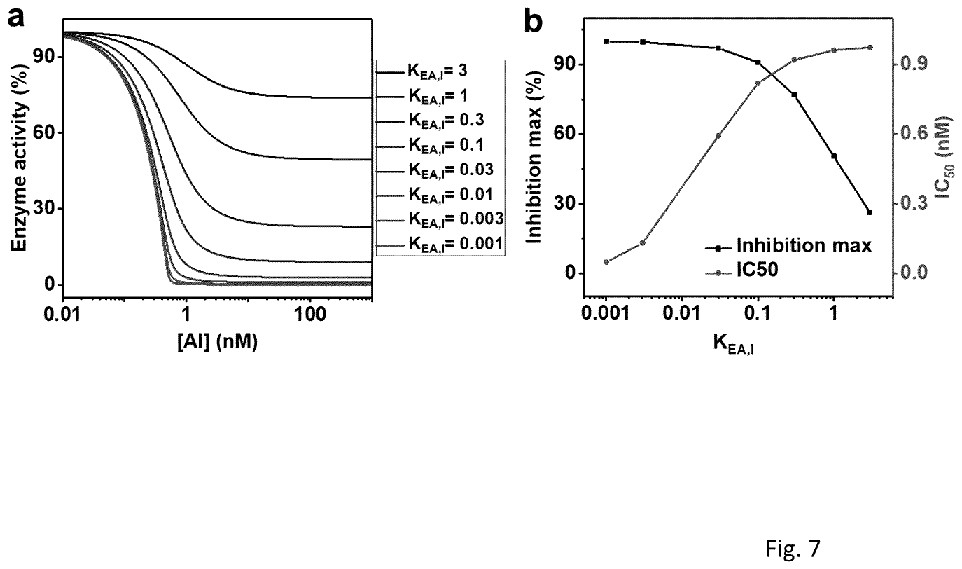

The increased potency of the EXACT inhibitor can be explained by a two-step thermodynamic binding model (FIG. 1d, methods, FIG. 7). In the first step, either the aptamer or small molecule moiety binds to the target protease with a dissociation constant presumably similar to the free aptamer or small molecule. This interaction positions the other moiety in proximity to its binding site, thus facilitating its binding. Notably, the second binding step is a unimolecular process and is independent of the EXACT inhibitor's concentration; this juxtaposition allows effective inhibition of the protease even at a very low inhibitor concentration.

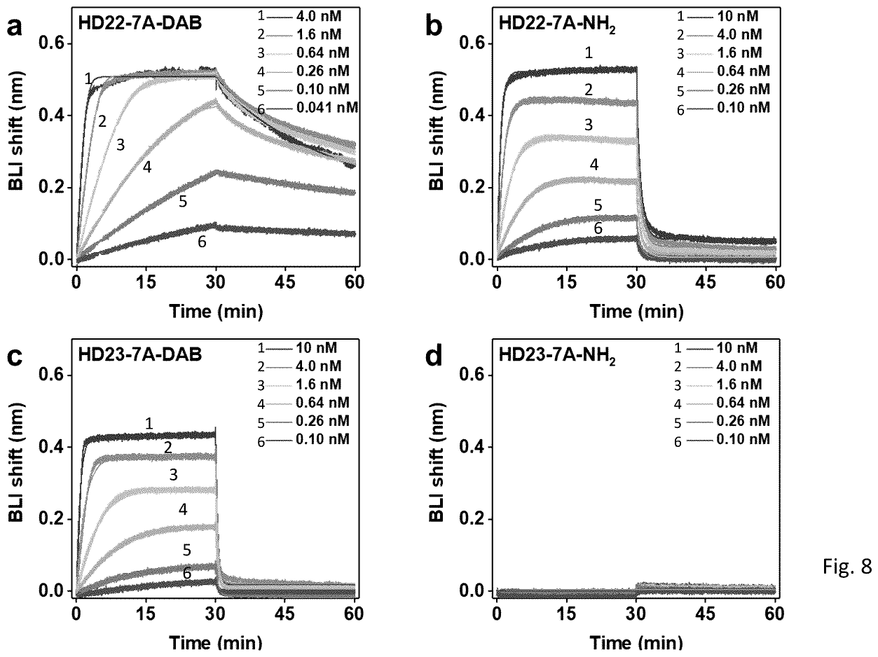

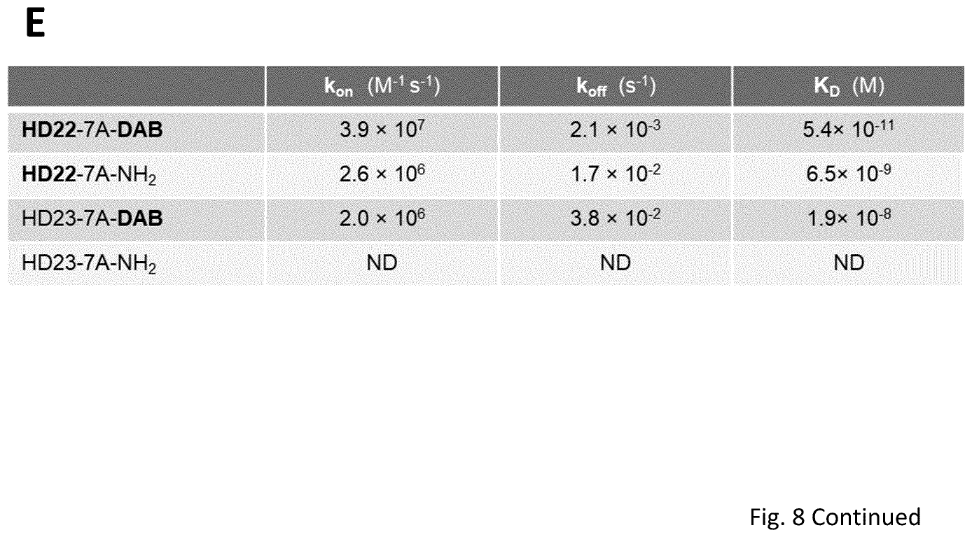

This binding model is supported by binding affinity and kinetic measurements of HD22-7A-DAB obtained via biolayer interferometry (BLI). EXACT inhibitor HD22-7A-DAB has a kₒₙ of 3.9×10⁷ M⁻¹s⁻¹ and a k_{off} of 2.1×10⁻³s⁻¹ for thrombin binding, yielding a K_{D} of 54 μM (FIG. 1e, FIG. 8). Such high affinity results from synergistic binding through two domains. When the DAB moiety is exchanged for an amine group (HD22-7A-NH₂), kₒₙ decreases by 15-fold while k_{off} increases by 8-fold, resulting in decreases in the K_{D} by over 100-fold compared to the intact EXACT inhibitor. Similar results are observed when the aptamer moiety is replaced with a non-binding point mutant aptamer (HD23-7A-DAB; FIG. 1e and FIG. 8).¹⁸ When both binding moieties are altered (HD23-7A-NH₂), no binding was observed (FIG. 1e and FIG. 8).

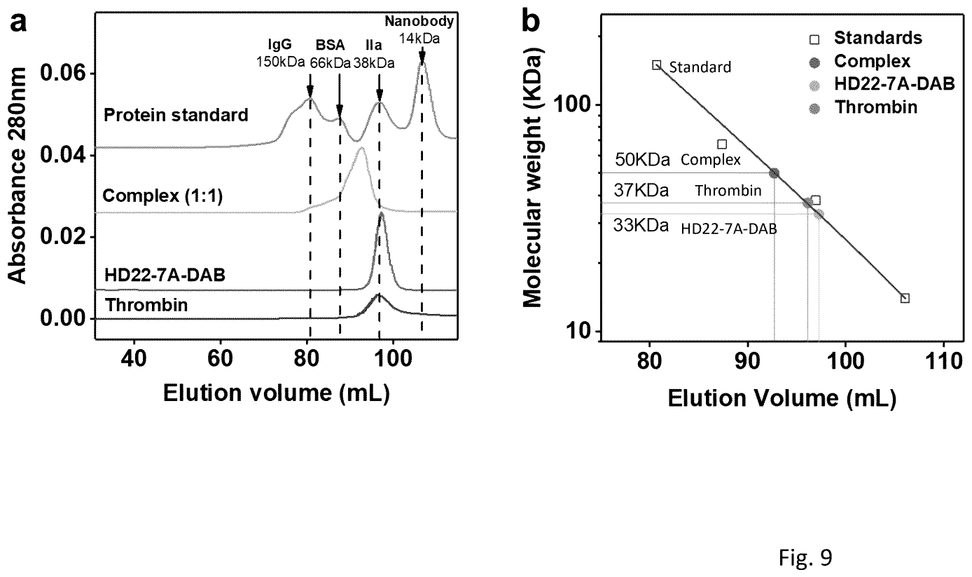

HD22-7A-DAB's two binding domains, in theory, allow it to bind two thrombin molecules, and, potentially, to induce the formation of complexes comprising multiple alternating thrombin and HD22-7A-DAB molecules. Therefore, the inventors characterized the binding stoichiometry of HD-22-7A-DAB by using size exclusion chromatography. The apparent molecular weight of thrombin and HD22-7A-DAB were determined to be 37 k and 30 k respectively. The inhibitor-thrombin complex yielded one major peak with Mr=50 k, consistent with the predominance of a 1:1 complex (FIG. 9). Moreover, the high affinity of the EXACT inhibitor for thrombin results in minimal dissociation of the components during the 180 minutes of chromatographic separation.

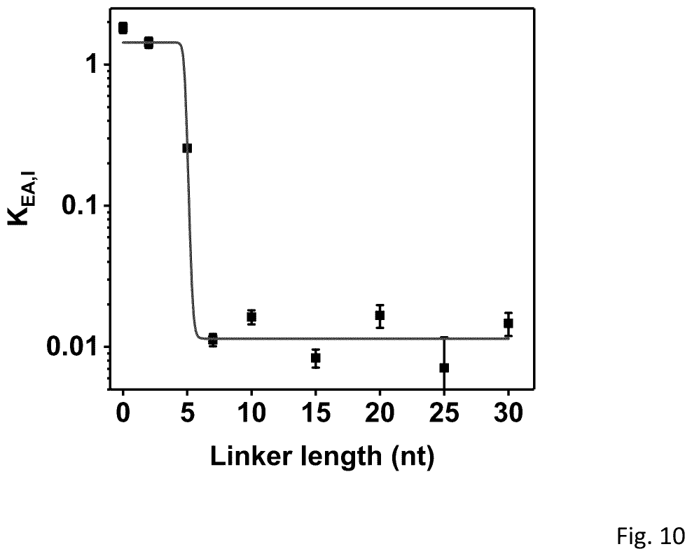

Synergistic-binding of HD22-7A-DAB can be tuned by varying linker length. According to the two-step binding model shown in FIG. 1D, the unitless dissociation constant of the second step, K_{EA,I}, greatly modulates the EXACT inhibitor's potency. Both the inhibitor's structure and its inhibitory mechanism suggested that changing the linker length could tune K_{EA,I}. To investigate that possibility, the inventors synthesized a series of HD22-DAB conjugates with linker lengths ranging from 0 to 30 nucleotides (nt) and tested their potency in the fluorescent peptidyl substrate cleavage assay (FIG. 1f). Even the conjugate with the shortest linker domain (0A, estimated length=8 Å) showed a high thrombin-binding affinity (IC₅₀=0.55±0.04 nM), probably due to the high affinity of the HD22 moiety.¹⁴ However, it only maximumly reduces thrombin activity by 55% (FIG. 1, f&g). Such a binding pattern is consistent with our proposed binding model. A K_{EA,I} of 1.8 indicated moderate synergy between the aptamer and small molecule moieties (FIG. 10), which could result from the deformation of the aptamer component to facilitate dabigatran binding. With increasing length of the linker, IC₅₀ and K_{EA,I} of the conjugate decreases, and the maximum inhibition increases, indicating more effective inhibition of thrombin, as the length and flexibility of the linker better accommodates bivalent-binding. When the linker length reaches 7-nt (estimated length 49 Å), the conjugate can fully inhibit thrombin (>99%) with an IC₅₀ of 0.10 nM. A K_{EA,I}˜0.01 indicated the aptamer and small molecule moiety almost always bind simultaneously. Further increasing of linker length to 30 nt (estimate length 18.5 nm) does not significantly alter the maximum inhibition, IC₅₀ or K_{EA,I} of the conjugate. Such results show that the strong synergy between the small molecule and the aptamer occurs over a broad range of linker lengths as long as the spacer can span than the distance between the active site and exosite, a critical consideration for designing additional EXACT inhibitors.

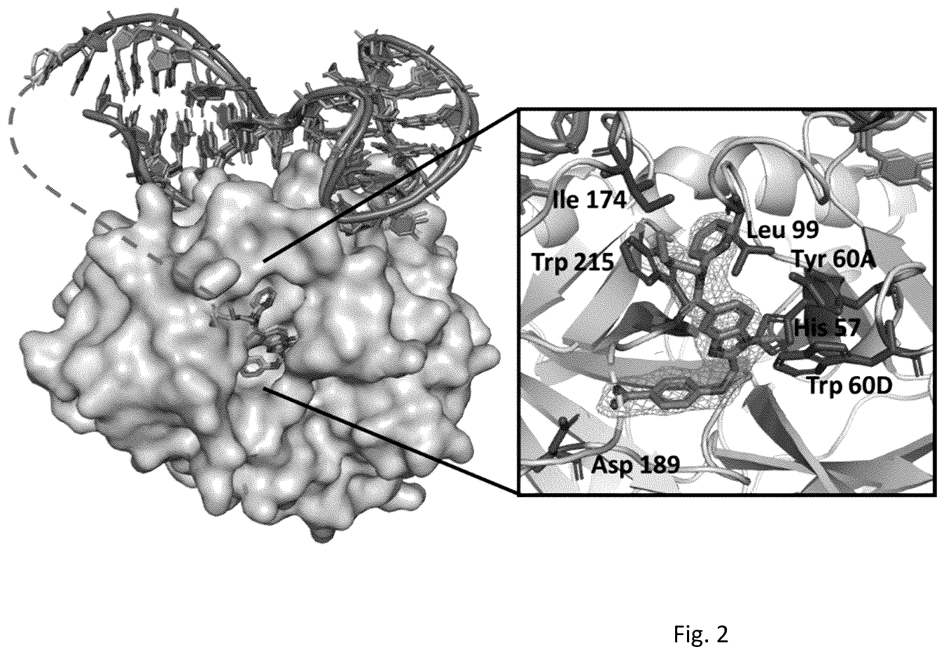

Structure analyses of HD22-7A-DAB binding to thrombin reveals hirudin-like binding mechanism of EXACT inhibitor. To further explore HD22-7A-DAB binding to and inhibition of thrombin, the inventors solved the x-ray structure of thrombin complexed with HD22-7A-DAB at 2.2 Å resolution (FIG. 2, Table 2). The diffraction data allows unambiguous placement of both aptamer and DAB moieties onto thrombin. The aptamer-thrombin interface in HD22-7A-DAB corresponds well to the previously determined structure of HD22 bound to thrombin.¹⁵ DAB is bound at the S1 specificity pocket where its amidine group is appropriately juxtaposed with Asp189, as found in the previously reported structure of the thrombin-DAB complex.¹³ The carboxyl group of DAB moiety is displaced from the reported thrombin-dabigatran complex possibly to accommodate the linker's position (FIG. 2). Only one deoxyadenine of the 7-nt linker at the 5′ terminus of HD22 is unambiguously resolved in the structure. At lower map contour levels, a partial second nucleotide is resolved, as is density potentially representing other nucleotides in the linker. These observations are consistent with the high flexibility of the linker as was proposed from the inhibition studies. The structure is also consistent with the EXACT inhibitor design wherein the aptamer and DAB moieties occupy the appropriate sites on thrombin as when present individually.

TABLE 2
Data collection and refinement statistics (molecular replacement)

| | HD22-7A-DAB-IIa | |
|---|---|---|
| | PDB ID: 8TQS | |
| Data collection | | |
| Wavelength (Å) | 0.9793 | |
| Space group | P 41 21 2 | |
| Cell dimensions | | |
| a, b, c (Å) | 81.63, 81.63, 190.51 | |
| α, β, γ (°) | 90, 90, 90 | |
| Resolution (Å) | 41.17-2.21 | (2.2-26) |
| R_{merge} | 0.127 | (1.3) |
| I/σI | 11.7 | (1.8) |
| Completeness (%) | 99.23 | (91.8) |
| Redundancy | 14.4 | (12.1) |
| Refinement | | |
| Resolution (Å) | 2.2 | |
| No. unique reflections | 33265 | (1614) |

| | |
|---|---|
| R_{work}/R_{free} | 0.204/0.24 |
| No. atoms | 31011 |
| Protein + RNA | 3000 |
| Ligand/ion | 63 |
| Water | 14 |
| B-factors | |
| Protein + RNA | 72.2 |
| Ligand/ion | 86.5 |
| Water | 62.4 |
| Clashscore | 7.62 |
| R.m.s. deviations | |
| Bond lengths (Å) | 0.011 |
| Bond angles (°) | 2.03 |
| Ramachandran | |
| Favored (%) | 96.85 |
| Allowed (%) | 3.15 |
| Outliers (%) | 0 |

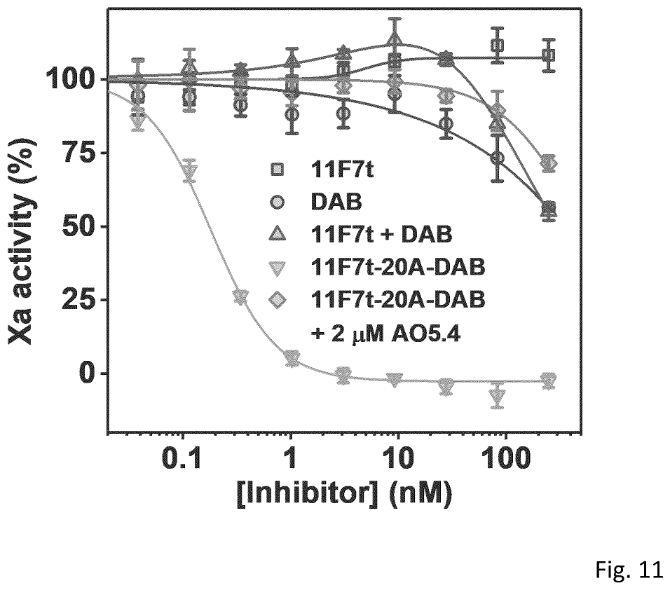

Exosite-binding aptamers can regulate the selectivity and antidote-mediated reversibility of EXACT inhibitors. The ability of an exosite-binding aptamer HD22 to promote binding of a small molecule DAB to thrombin's active site prompted us to test if another exosite-binding aptamer can be used to manipulate the inhibition potency and selectivity of this small molecule inhibitor. It is well known that DAB is a fairly selective thrombin active site inhibitor, although it also binds several other serine proteases, such as factor Xa, but with ˜1000-fold weaker affinity.¹³ The inventors therefore conjugated DAB to the 5′ end of a 36-nt factor Xa RNA aptamer 11F7t via a 20 nucleotide poly 2′ O-methyl A linker (Table 1).¹⁹ Linker length was chosen to be in excess of the distance required to span the distance between the 5′ terminus of 11F7t and the S1 site of factor Xa based on the crystal structure of factor Xa complexed with the aptamer²⁰. This EXACT inhibitor, termed 11F7t-20A-DAB, was evaluated in the fluorescent peptidyl substrate cleavage assay along with several control derivatives. As expected, DAB only weakly inhibits factor Xa (IC₅₀>500 nM) (FIG. 1h). The unconjugated aptamer 11F7t also showed no inhibition as it does not impede access to the active site of the protease (FIG. 11)¹⁹. However 11F7t-20A-DAB achieved an IC₅₀ 0.18 nM, which indicates a higher affinity than the free aptamer (K_{D}=1.26 nM) for factor Xa.¹⁹ The >2500-fold enhancement in the IC₅₀ for factor Xa when DAB is conjugated to 11F7t but not to HD22, illustrates how the exosite binding aptamer increases affinity for the otherwise weak binding of DAB to the active site of factor Xa to yield a potent EXACT inhibitor. Consistent with this reasoning, the 11F7t-20A-DAB inhibitor has a thrombin inhibition profile that was no different from DAB alone and much weaker potency than that obtained for factor Xa inhibition (FIG. 1i).

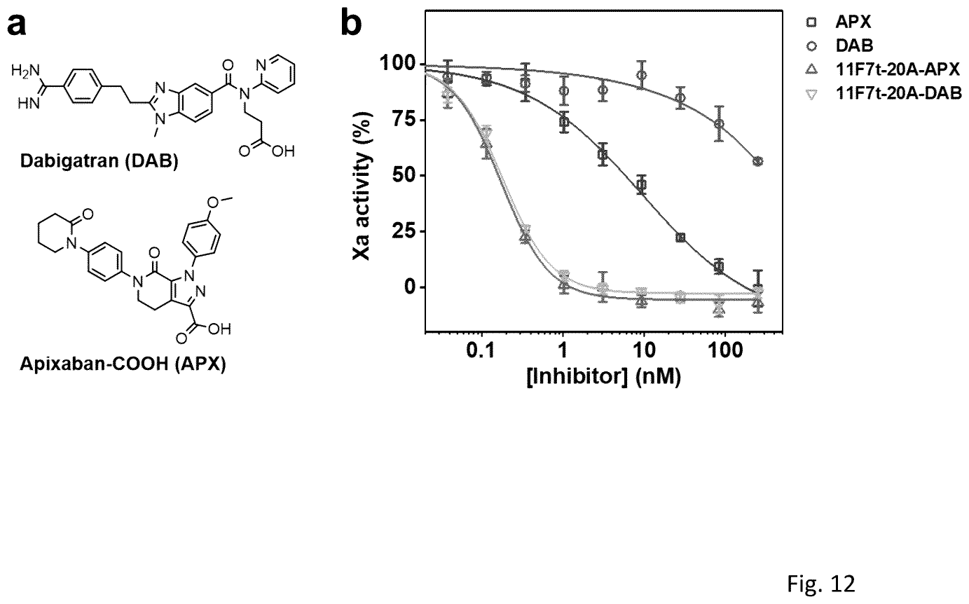

Remarkably, the active-site inhibitor binding appears to become a concentration-independent unimolecular process upon aptamer binding. Therefore, the potency of the EXACT inhibitor is largely independent of the affinity of the active-site inhibitor. For example, when the weak-binding DAB moiety of 11F7t-20A-DAB is replaced with a strong-binding factor Xa inhibitor apixaban-COOH (APX, IC₅₀=5.5 nM), the resulting 11F7t-20A-APX achieved almost identical potency against factor Xa (IC₅₀=0.17 nM) as 11F7t-20A-DAB (IC₅₀=0.18 nM) (Table 1, FIG. 12). These results clearly illustrate the versatility of the exosite targeting aptamer, as it can redirect inhibition of a small molecule inhibitor against a totally different enzyme, allowing generation of selective and potent EXACT inhibitor regardless of the inherent selectivity and potency of the small molecule active-site inhibitor.

One of the valuable traits of aptamer-based inhibitors is that they can be reversed by complementary antidote oligonucleotides (AO) that disrupt the folded structure of the aptamer.²¹ The inventors next investigated if the activity of the aptamer-based EXACT inhibitor HD22-7A-DAB can be reversed using an AO. Strikingly, an AO termed AO2 (Table 1) that is complementary to the entire length of the aptamer and the linker region reduced the anticoagulant potency of HD22-7A-DAB by more than 10,000-fold (FIG. 1c), despite HD22-7A-DAB's high affinity for thrombin. Notably, although AO2 does not directly interact with the DAB moiety, the antidote reduces the potency of the EXACT conjugate to an extent that is significantly lower than free DAB (FIG. 1c). This result illustrates the fundamental role of the exosite-binding component of the EXACT inhibitor in driving affinity for active site inhibition. In line with the importance of the worm-like flexible behavior for the single stranded DNA linker, hybridization of the AO to the entire length of the aptamer plus linker not only disrupts exosite-dependent binding to thrombin but stiffens the linker by forming double-stranded DNA.^{22,23} This alteration, in addition to steric effects, is likely a partial explanation for why addition of AO to HD22-7A-DAB restores thrombin activity to a level significantly higher than thrombin activity inhibited with free DAB. (FIG. 1c and FIG. 13). Testing this concept, the inventors evaluated an antidote lacking two nucleotides that hybridize with the 5′ terminus of the linker region (the last two “T” nucleotides of AO2 and called AO1). This AO (AO1) resulted in considerably reduced antidote efficacy. By contrast, truncation of the antidote's aptamer binding domain up to 12 nucleotides does not significantly reduce antidote efficacy (Table 1 and FIG. 13, shown as AO3-AO5). With this insight, the inventors developed an antidote for 11F7t-20A-DAB (AG5.4) that pairs with the entire linker region and part of the aptamer region and observed that it effectively reverses the inhibition of the EXACT inhibitor against factor Xa (Table 1 and FIG. 11).

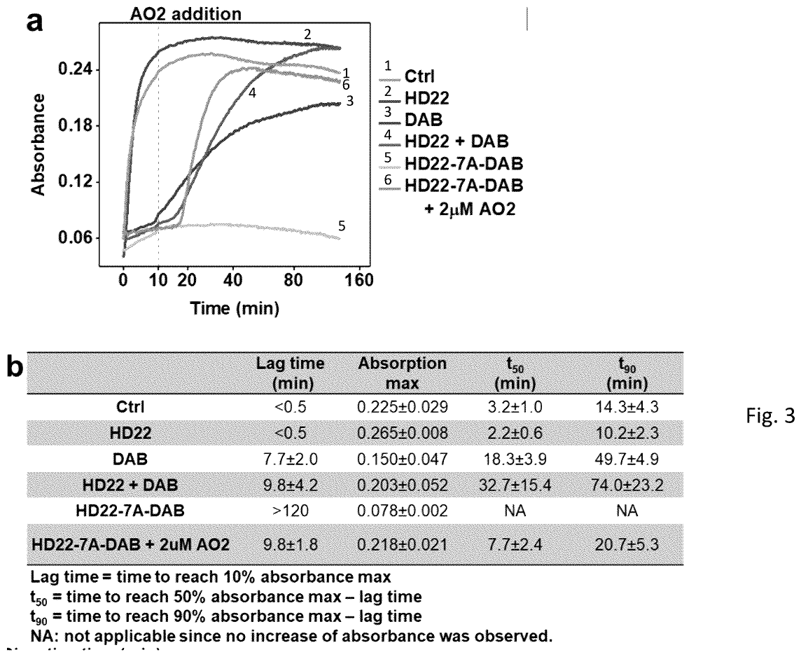

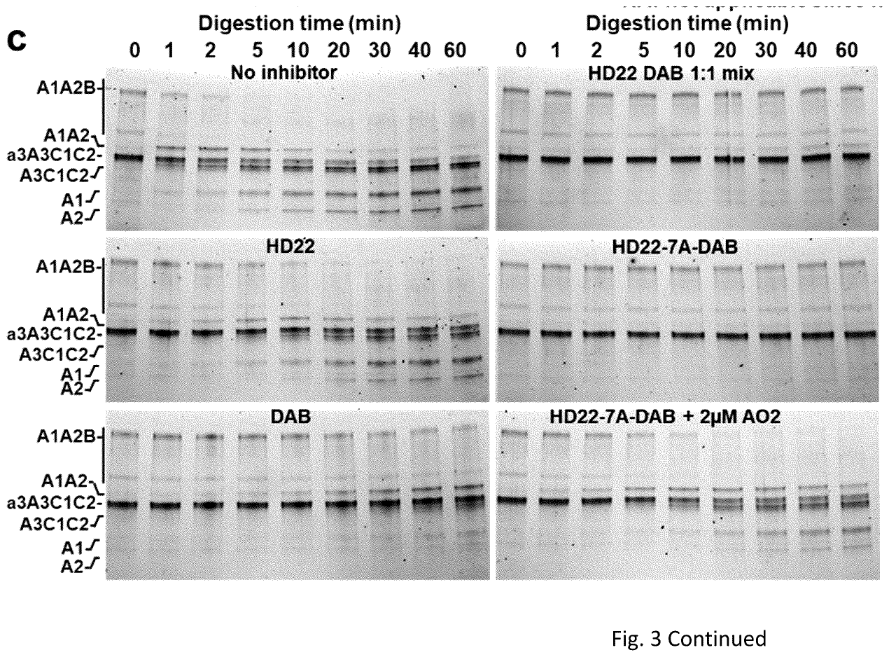

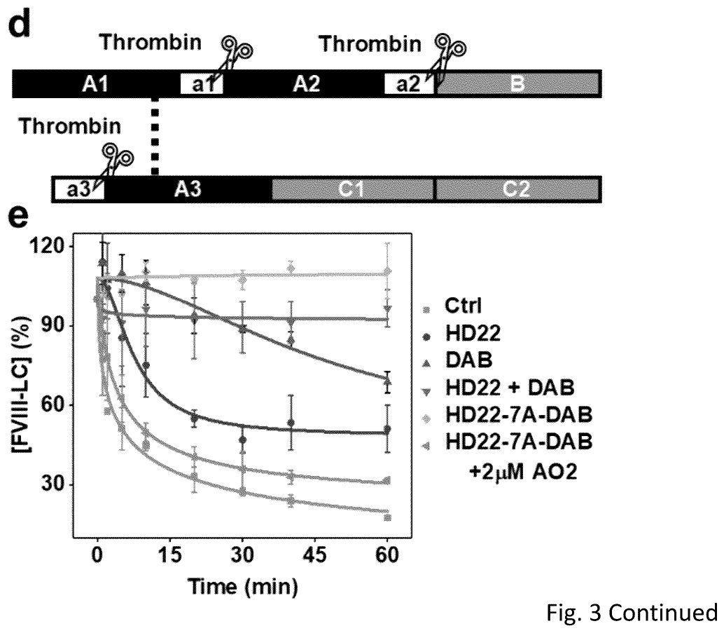

EXACT inhibitors impede multiple functions of thrombin. Thrombin has two exosites that mediate its enzymatic activity on multiple substrates, including fibrinogen, factors V, VIII, XI and XIII. The inventors investigated how EXACT inhibitor HD22-7A-DAB impacts thrombin exosite-mediated cleavage of its natural substrates. Thrombin's cleavage of fibrinogen was characterized by a fibrin turbidity assay.²⁴ In the absence of an inhibitor, thrombin rapidly cleaves fibrinogen, leading to the formation of fibrin clots. Light scattering by the fibrin clots results in increased absorbance at 550 nm wavelength (FIG. 3a). DAB inhibits thrombin activity, resulting in a 7.7 min delayed onset of clot formation, a slower clot formation, and lower maximum absorbance (FIG. 3b). As expected, HD22 did not inhibit this thrombin activity, since it binds exosite II and does not block fibrinogen-thrombin interaction or thrombin's active site.²⁵ In the presence of 50 nM EXACT inhibitor HD22-7A-DAB however, no significant absorbance increase was observed during the course of the experiment (120 minutes), indicating that thrombin's enzymatic activity on fibrinogen is completely inhibited. An equimolar mixture of HD22 and DAB did not yield a comparable potency to the EXACT inhibitor, highlighting the power of bimodal binding. To test the efficacy and kinetics of antidote reversal, AO2 was added to the thrombin-fibrinogen-HD22-7A-DAB mixture at the 10-minute time point. After a 9.8-min lag time, the absorbance of the AO2-containing solution rapidly increased and saturated over 20.7 minutes (FIG. 3a&b). These results demonstrate that the antidote can rapidly and effectively reverse the activity of the EXACT conjugate.

The inventors then used SDS-PAGE analysis to characterize the effect of HD22-7A-DAB on FVIII activation which is mediated by thrombin ABE2 (FIG. 3c-e). Thrombin cleavage of FVIII results in the disappearance of both heavy and light chain of FVIII and appearance of several cleaved products (FIG. 3c&d). The presence of 100 nM DAB delayed such digestion. As expected, HD22 also showed an inhibitory effect due to its binding to ABE2. In the presence of HD22-7A-DAB, no proteolysis was observed for over one hour, demonstrating a much higher potency than DAB, HD22, or their equimolar mixture (FIG. 3c&e).

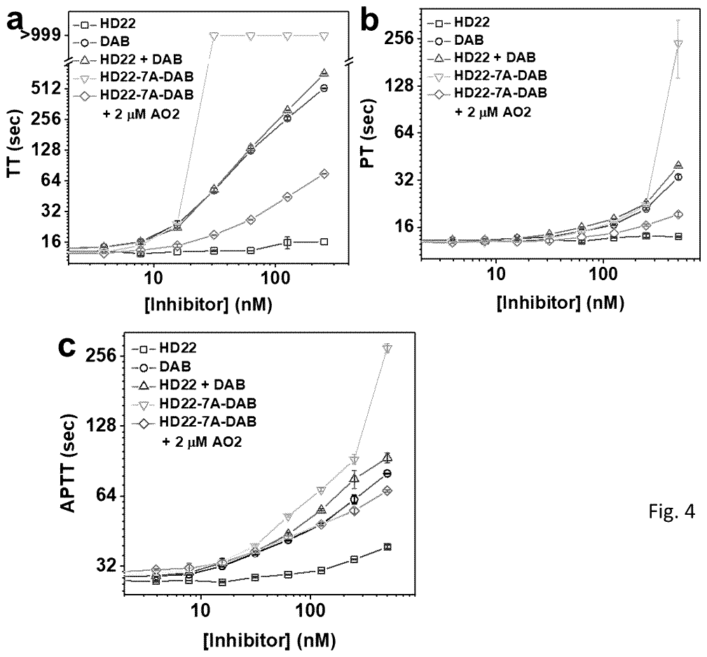

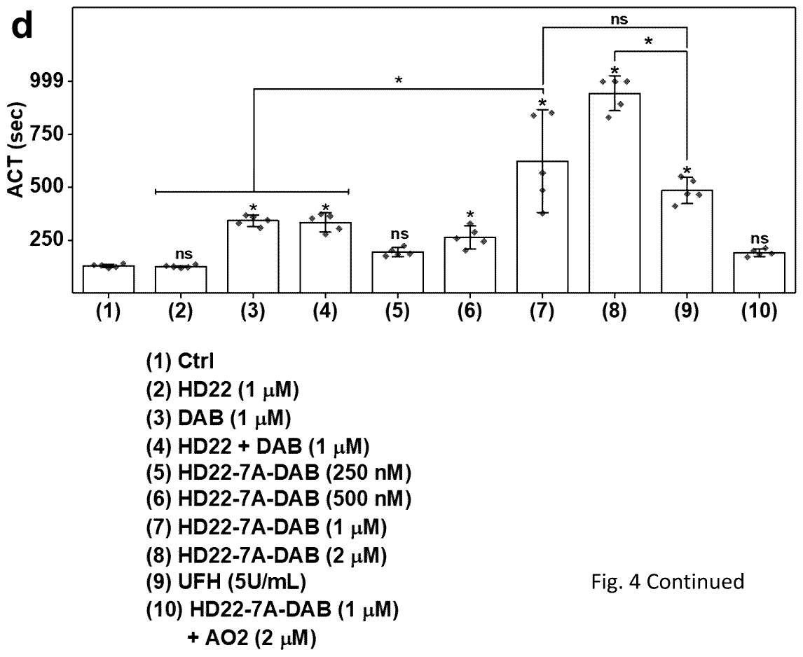

Potent anticoagulation achieved by EXACT inhibitor HD22-7A-DAB. The inventors then investigated the anticoagulation activity of HD22-7A-DAB in human plasma with a series of clinical clotting assays. Thrombin time (TT) directly probes the terminal step of the coagulation cascade, in which fibrinogen is cleaved by thrombin to form fibrin clots. As expected, free HD22 minimally affected TT as thrombin exosite II is not involved in fibrinogen cleavage (FIG. 4a). DAB, on the other hand, dose-dependently prolonged TT, resulting in a clotting time of 518 sec at the concentration of 250 nM. An equimolar mixture of HD22 and DAB prolonged TT to a similar extent as DAB alone. HD22-7A-DAB showed a different dose-dependent effect on TT compared to both DAB and free HD22; when the EXACT inhibitor concentration is lower than the added thrombin (13.4 nM), the conjugate showed limited effect on TT. However, once the EXACT inhibitor's concentration exceeded thrombin's concentration, TT dramatically increased and surpassed 999 seconds at a concentration of 31 nM. Notably, the high anticoagulant potency of HD22-7A-DAB can be largely reversed within 5 minutes by the addition of 2 μM AO2.

The prothrombin time (PT) and activated partial thromboplastin time (aPTT) assays were used to assess the effect of HD22-7A-DAB on the extrinsic and intrinsic coagulation pathways, respectively (FIG. 4, b&c). At a concentration of 500 nM, the EXACT inhibitor prolonged the PT and aPTT significantly longer than the same concentration of DAB. HD22 alone produced no anticoagulant effect, and equimolar mixtures of DAB and HD22 prolonged the PT and aPTT to an extent comparable to that of DAB alone. The anticoagulant effect of HD22-7A-DAB on both assays can also be efficiently reversed by AO2. However, HD22-7A-DAB could not completely block clotting in these assays because the EXACT inhibitor cannot prohibit activation of thrombin by factor Xa. Thus, HD22-7A-DAB will lose its effectiveness when the thrombin concentration eventually exceeds the concentration of the conjugate. The inventors also tested the anticoagulation activity of factor Xa inhibitor 11F7t-20A-APX in aPTT assay and showed that the EXACT inhibitor showed significantly higher anticoagulant activity than free aptamer, small molecule inhibitor, and their equimolar mixture.

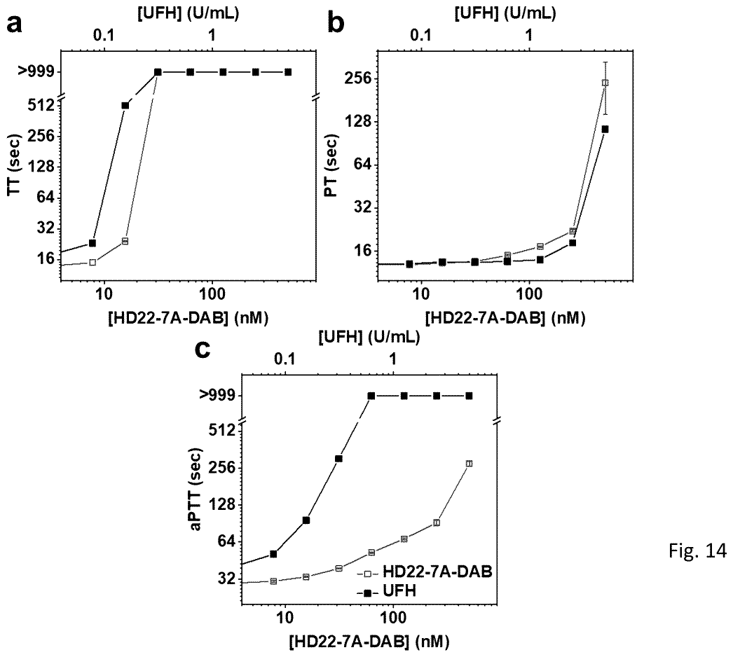

The inventors further compared the anticoagulant effect of HD22-7A-DAB to that of unfractionated heparin (UFH) the most potent clinical anticoagulant. UFH activates the anticoagulant protein antithrombin, which then irreversibly inactivates multiple procoagulant proteases, particularly thrombin, factor Xa, and factor IXa, and results in high anticoagulant activity.²⁶ HD22-7A-DAB shows potency that rivals UFH in TT and PT assays (FIG. 14), which can be attributed to its ultra-high thrombin binding affinity. Not surprisingly, HD22-7A-DAB is a weaker anticoagulant than UFH in the aPTT assay, as HD22-7A-DAB does not inhibit factors IXa and Xa, two proteases that significantly affect aPTT.

Finally, the inventors assessed the anticoagulation activity of HD22-7A-DAB in whole human blood with the activated clotting time (ACT), a point of care assay commonly utilized to monitor the level of anticoagulation in patients during cardiopulmonary bypass (CPB) and other invasive procedures that require rapid onset systemic anticoagulation.²⁷ HD22-7A-DAB dose dependently prolonged ACT (FIG. 4d). At 1-μM concentration, the ACT increased to 624 sec, significantly higher than the ACTs that followed the addition of DAB, HD22, or their equimolar mixture at the same concentration. More importantly, HD22-7A-DAB at 1 μM and 2 μM doses were able to prolong ACT to a level comparable to and significantly higher than 5 U/mL UFH, the standard dose employed during CPB, respectively. Those findings indicate an anticoagulant potency in whole blood that can potentially support highly thrombogenic procedures, including CBP. Thus HD22-7A-DAB is the first aptamer-based, de novo generated EXACT inhibitor that can achieve such profound anticoagulant activity.

### CONCLUSION

Small molecule active site inhibitors have proven incredibly clinically valuable to study and modulate enzymes involved in many human diseases; their use to target coagulation factors to treat and prevent cardiovascular disease and stroke has been particularly impactful. However, developing such inhibitors with both high target affinity and selectivity can be challenging given the low binding surface of the small molecules and the similarity among the active sites on different but related enzymes. Inspired by hirudin and other anticoagulants that targeting both EXosite and ACTive sites of key coagulation proteases, the inventors show that this natural concept can be readily incorporated into rational drug design using chemically synthesized binding agents, which yield a new generation of potent and rapidly reversible, EXACT inhibitors that distinguish between structurally homologous enzymes, such as thrombin and factor Xa.

Strikingly and surprisingly, the inventors discover that the binding properties of the active site-binding small molecule can be greatly modulated by the exosite-binding aptamer to which it is attached in three ways. First, the bimodal binding mechanism greatly increases the protease accessibility and potency of the active site-binding moiety. Second, the high synergy between the exosite binding aptamer with the small molecule even allows one to manipulate the small molecule's selectivity. Third, the reversal of active site inhibition by an antidote against the exosite-binding aptamer is particularly noteworthy as hemorrhage remains the chief safety issue associated with commonly utilized active site-targeted antithrombotic agents.

Aptamers in the past few decades have struggled to find their therapeutic niche and distinguish themselves from antibodies, small molecules, and other classes of therapeutics^{28 29}. However, the ease with which oligonucleotides can be chemically conjugated to small molecules and the ability of aptamers to selectively bind exosites on enzymes^{18-20,30-39} makes them particularly suitable for this type of rational drug design. Our results indicate that potent, selective, and reversible EXACT inhibitors can be created for virtually any enzyme by appropriately linking an exosite-binding aptamer to even a low affinity, low specificity, small molecule active site inhibitor. Thus, this generalizable approach may also help resuscitate many small molecule drugs abandoned because they were not effective or selective enough on their own.

### Methods

Materials: All DNA Oligos were purchased from Integrated DNA Technologies Inc. Modified RNA oligos were synthesized in house using a Mermade oligo synthesizer followed by HPLC purification. Chemicals were purchased from Sigma without specification. Dabigatran (DAB) and apixaban-COOH (APX) were purchased from AK Scientific Inc. Human alpha thrombin, human factor Xa, and human fibrinogen were purchased from Haematologic Technologies Inc. Recombinant human factor VIII was purified from Kogenate FS (Bayer) and quantified by UV abosorbance.

Synthesis and purification of ADIC with poly (A) linker: EXACT inhibitors were synthesized via EDC/NHS using a reported method with modification.⁴⁰ Briefly, 1 μmole of EDC and 4 μmole of NHS were mixed in 22 μL of H₂O/DMSO solution (v:v=15%:85%). The mixture was immediately added to 230 μL of 5.5 mM DAB or APX dissolved in DMSO. After 30 min of incubation at room temperature, 5 nmole of amine-modified aptamer derivatives dissolved in 60 L of 420 mM TEA/HCl buffer, pH 10 were added into the activated DAB solution and incubated at room temperature overnight. The unreacted DAB was removed by ethanol precipitation, and the aptamer-DAB conjugate was purified from the unreacted aptamer and other residue impurities by HPLC and verified by IES-MS. The purity of the final product was validated on a 15% denaturing PAGE (FIG. 5).

Fluorogenic activity assay: All reagents were freshly reconstituted in the reaction buffer (20 mM HEPES, 150 mM NaCl, and 2 mM CaCl2), 0.02%, Tween 20, pH 7.5) before the assay. 10 μL of thrombin or factor Xa (final concentration 0.5 nM) were first incubated with 5 μL of inhibitor, inhibitor/antidote mixture, or buffer control on a 384-well opaque plate for 5 min at 28° C. 10 μL of fluorogenic substrate (final concentration 50 μM) was then added to the mixture and the time-course fluorescence (λₑₓ=352 nm, λₑₘ=470 nm) of the sample were recorded every minute for 15 min at 28° C. using a SpectraMax i3 microplate reader (Molecular Devices). The catalytic rate of the protease was quantified by the slope from linear regression of the time-dependent fluorescence intensity and normalized with the sample containing no inhibitor as 100%. Each experiment was performed in triplicates. In the fluorogenic substrate assay, IC₅₀ instead of Kᵢ is compared due to the different inhibition mechanism between exosite and active site binding inhibitors. IC₅₀ of an active site inhibitor is higher than its Kᵢ due to the competition between the inhibitor and the substrate. One the other hand, IC₅₀ of an exosite inhibitor is similar to its Kᵢ. The inventors believe that IC₅₀, compared to Kᵢ, better represents the potency of different inhibitors in the presence of substrate.

Bio-layer interferometry: Bio-layer inferometry was performed using Octet R8 BLI System (Sartorius). Briefly, 3.3 nM of biotinylated HD22-7A-DAB, HD22-7A-NH₂, HD23-7A-DAB, and HD23-7A-NH₂ were immobilized to Octet streptavidin biosensors (Sartorius). The baseline was collected in buffer (20 mM HEPES, 150 mM NaCl, and 2 mM CaCl₂), 100×BSA, pH 7.5) followed by a 30-min association in buffer containing different concentrations of thrombin. A 30-min dissociation was performed in buffer containing 200 nM HD22-7A-DAB. The existence of free HD22-7A-DAB in dissociation buffer prevents re-binding of thrombin and provides more accurate k_{off} measurements.

Size exclusion chromatography: All experiments were performed using a BioCADRPM perfusion chromatography workstation with a HiLoad 16/600 Superdex 200 pg column (cytiva). Before experiment, the column was equilibrated overnight in buffer (20 mM HEPES, 150 mM NaCl, and 2 mM CaCl₂), 0.1%, PEG8000, pH 7.5). 1.25 nmole of thrombin, HD22-7A-DAB, or their equimolar mixture in 250 μL buffer was loaded to the column with a buffer elution rate of 0.5 mL/min for at least 240 min. The absorbance at 260 nm was recorded throughout the experiment. The protein standard consisted of human IgG (150 KDa), BSA (67 KDa), thrombin S195A (38 KDa), and a nanobody (14 KDa).

X-ray crystallography: A mixture of 150 mM IIa_{S195A} and 160 mM HD22-7A-DAB in 20 mM HEPES, 0.15 M NaCl, pH 7.4 was mixed with an equal volume of 0.1 M MES monohydrate, pH 6.0, 22% (v/v) polyethylene glycol 400 and crystals were grown from 2 ml sitting drops by vapor diffusion. X-ray diffraction data were collected at beamline 17-ID-1 (AMX) at NSLS-II. Data were merged and scaled using the AutoProc pipeline⁴¹ using XDS,⁴² Aimless,⁴³ Pointless⁴⁴ and StarAniso. Molecular replacement was done using the human thrombin structure 1PPB⁴⁵ and the structure of HD22 from 5EW1⁴⁶ using Phenix.Phaser.⁴⁷ Initial rounds of model completion and refinement were done with COOT⁴⁸ and Phenix.Refine.⁴⁷ The last round of refinement was done using PDBREDO.⁴⁹

Fibrinogen turbidity assay: All reagents were freshly reconstituted in the reaction buffer the assay. 20 μL Thrombin (final concentration 2.5 nM) was incubated with 10 μL thrombin inhibitors (final concentration 50 nM) or buffer control at 37° C. for 5 min. 20 μL fibrinogen (final concentration 0.8 mg/mL) was then added to the reaction and absorbance at 550 nm was measured every 30 seconds over 130 min using a SpectraMax i3 microplate reader to monitor clot formation. To test the kinetics of antidote reversal, 1 μL AO2 (final concentration 2 μM) was added to the reaction 10 min after fibrinogen addition. The maximum absorption over the period of assay in the absence and presence of different inhibitors were determined. The time to reach 10%, 50%, and 90% maximum absorption increase from the start point were calculated and recorded as lag time, t₅₀, and t₉₀, respectively, for each inhibitor to evaluate the kinetic of fibrinogen activation. Each experiment was performed in triplicates to determine the mean and standard deviation of each parameter.

Thrombin cleavage of FVIII: All reagents were freshly reconstituted in the reaction buffer the assay. Thrombin (final concentration 1 nM) was incubated in the absence or presence of thrombin inhibitors (final concentration 100 nM) at 37° C. for 5 min following by addition of recombinant human FVIII (final concentration 100 nM). Sample were collected at 1, 2, 5, 10, 20, 30, 40, and 60 min of reaction and quenched in SDS loading buffer and heating at 95° C. for 5 min. The digestion products at different time points are then characterized on a 4-20% PAGE gel. Thrombin's activity on activating FVIII in the absence and presence of thrombin inhibitors were quantified using the time-course concentration of intact light chain under thrombin digestion determined by band intensity. The assay was performed in duplicates.

Plasma coagulation assays: Thrombin time (TT), prothrombin time (PT), and activated partial thromboplastin time (aPTT) assays were performed in citrated normal human plasma on a hemostasis coagulation analyzer (Diagnostica Stago). For TT, 5 μL of inhibitors in the reaction buffer were mixed with 100 μL of plasma and incubated at 37° C. for 5 min. 50 μL of thrombin (6 NIH units/ml) in the reaction buffer was then added to initiate clotting. For PT, 5 μl of inhibitors in the reaction buffer were mixed with 50 μL of plasma and incubated at 37° C. for 5 min. 100 μL of TriniCLOT PT Excel S reagent was then added to initiate clotting. For aPTT, 5 μL of inhibitors in the reaction buffer were mixed with 50 μL of plasma and incubated at 37° C. for 5 min, 50 μL of TriniCLOT aPTT S reagents was then added followed by another 5 min incubation at 37° C. Finally, 50 μL of 20 mM CaCl₂) was added to initiate clotting. To characterize antidote reversal of HD22-7A-DAB in the above assays, 5 μL AO2 (finial concentration 2 μM) in the reaction buffer was added after 5-min incubation between plasma and HD22-7A-DAB, followed by another 5 min of incubation before the next step. All assays are performed induplicates.

Active clotting time (ACT): Citrated blood (72 μL) freshly collected from heathy donors were incubated with 6 μL inhibitors reconstituted in the reaction buffer at room temperature for 3 min following addition of 2.1 μL CaCl₂) (245 mM). The blood mixture was then immediately analyzed on an ACT+ cuvette (Accriva Diagnostics) using a Hemochron Jr Signature (Instrumentation Laboratory). The assay was performed with a N value of five. One-way ANOVA test was used to compare between two sets of data.

Binding model of EXACT inhibitors. The following two-step model describes the binding equilibriums between a bivalent EXACT inhibitor with the protease. FIG. 1d represents the two possible pathways for the stepwise ligation of thrombin (E) with the EXACT inhibitor (AI) containing the aptamer (A) conjugated to DAB (I) by a linker. K_{E,A} and K_{E,I} represent the equilibrium dissociation constants for the initial ligation of either the aptamer or DAB to thrombin. As supported by the data, these binding constants are assumed to be equivalent to those seen with the monovalent ligands. Initial binding is followed by a dimensionless unimolecular step in which either I binds to the active site when A is already bound or A binds to the exosite when I is already bound to give a common doubly ligated product (AEI). As defined, AEI is increasingly favored at smaller values K_{EA,I}.

The relationship between the concentrations of E, AI, AIE, EAI, and AEI can be determined by three dissociation constants, K_{E,A}, K_{E,I}, and K_{EA,I} with following equilibriums:

$$AIE = \frac{E \cdot AI}{{K}_{E , A}} ; EAI = \frac{E \cdot AI}{{K}_{E , I}} ; AEI = \frac{E \cdot AI}{{K}_{E , A} \cdot {K}_{E A , I}}$$

With a given total concentration of protease and EXACT inhibitor to be Eₜ and AIₜ, respectively:

$${E}_{t} = E + A I E + E AI + AEI = E \cdot AI \cdot ( \frac{1}{A I} + \frac{1}{{K}_{E , A}} + \frac{1}{{K}_{E , I}} + \frac{1}{{K}_{E , A} \cdot {K}_{E A , I}} ) and$$

$$\begin{matrix}A {I}_{t} = AI + A I E + E AI + AEI \\ = AI + {E}_{t} - E \\ = \frac{A I + [ ( \frac{1}{{K}_{E , A}} + \frac{1}{{K}_{E , I}} + \frac{1}{{K}_{E , A} \cdot {K}_{E A , I}} ) \cdot AI ] \cdot ( AI + {E}_{t} )}{1 + ( \frac{1}{{K}_{E , A}} + \frac{1}{{K}_{E , I}} + \frac{1}{{K}_{E , A} \cdot {K}_{EA , I}} ) \cdot AI}\end{matrix}$$

From the above equations, AI can be determined as:

$$\frac{\begin{matrix}\sqrt{\begin{matrix}{[ ( \frac{1}{{K}_{E , A}} + \frac{1}{{K}_{E , I}} + \frac{1}{{K}_{E , A} \cdot {K}_{EA , I}} ) \cdot ( {E}_{t} - {AI}_{t} ) + 1 ]}^{2} + \\ 4 \cdot ( \frac{1}{{K}_{E , A}} + \frac{1}{{K}_{E , I}} + \frac{1}{{K}_{E , A} \cdot {K}_{EA , I}} ) \cdot {AI}_{t}\end{matrix}} - \\ [ ( \frac{1}{{K}_{E , A}} + \frac{1}{{K}_{E , I}} + \frac{1}{{K}_{E , A} \cdot {K}_{EA , I}} ) \cdot ( {E}_{t} - {AI}_{t} ) + 1 ]\end{matrix}}{2 \cdot ( \frac{1}{{K}_{E , A}} + \frac{1}{{K}_{E , I}} + \frac{1}{{K}_{E , A} \cdot {K}_{EA , I}} )}$$

Assuming that E, AIE, EAI, and AEI have activity of 1, α₁, α₂, and α₃, respectively, the relative activity of the protease (A) can be described as:

$$\begin{matrix}A = \frac{E}{{E}_{t}} + {α}_{1} \cdot \frac{AIE}{{E}_{t}} + {α}_{2} \cdot \frac{EAI}{{E}_{t}} + {α}_{3} \cdot \frac{AEI}{{E}_{t}} \\ = ( \frac{1}{AI} + \frac{{α}_{1}}{{K}_{E , A}} + \frac{{α}_{2}}{{K}_{E , I}} + \frac{{α}_{3}}{{K}_{E , A} \cdot {K}_{EA , I}} ) / \\ ( \frac{1}{AI} + \frac{1}{{K}_{E , A}} + \frac{1}{{K}_{E , I}} + \frac{1}{{K}_{E , A} \cdot {K}_{EA , I}} )\end{matrix}$$

And is a function of Alt with a given Eₜ, K_{E,A}, K_{E,I}, K_{EA,I}, α₁, α₂, and α₃. Inhibition max can be determined by 1-A when IJ approaches infinity:

$$( \frac{{α}_{1}}{{K}_{E , A}} + \frac{{α}_{2}}{{K}_{E , I}} + \frac{{α}_{3}}{{K}_{E , A} \cdot {K}_{EA , I}} ) / ( \frac{1}{{K}_{E , A}} + \frac{1}{{K}_{E , I}} + \frac{1}{{K}_{E , A} \cdot {K}_{EA , I}} )$$

And IC₅₀ can be determined as IJ that resulted inhibition halfway towards inhibition max:

$${IC}_{50} = 1 / ( \frac{1}{{K}_{E , A}} + \frac{1}{{K}_{E , I}} + \frac{1}{{K}_{E , A} \cdot {K}_{E A , I}} )$$

In FIG. 1f, K_{EA,I} of different inhibitors were calculated by global fitting of their inhibition curves with several assumptions:
1. K_{AIE} is shared between all EXACT inhibitors.
2. K_{EAI} is shared between all EXACT inhibitors and is equivalent to the IC₅₀ of free DAB (50 nM) in the same experimental setting.
3. Eₜ is shared between all EXACT inhibitors as all experiments were performed with same thrombin concentration.
4. α₁, α₂, and α₃ were determined by the relative activity of thrombin in the presence of saturating concentration of HD22, DAB, and HD22-7A-DAB, to be 0.70, 0, and 0, respectively

In FIG. 7, the inventors further simulated enzyme inhibition curves with different K_{EA,I} (ranging from 0.001 to 3), with the additional assumptions that K_{E,A}=1 nM, K_{E,I}=50 nM, α₁=1, α₂=0, α₃=0, and Eₜ=0.5 nM. Which demonstrated a very similar pattern to experimental data shown in FIG. 1f & g.

### REFERENCES

1 Ledizet, M., Harrison, L. M., Koskia, R. A. & Cappello, M. Discovery and pre-clinical development of antithrombotics from hematophagous invertebrates. Curr Med Chem Cardiovasc Hematol Agents 3, 1-10, doi:10.2174/1568016052773315 (2005).
2 Rydel, T. J. et al. The structure of a complex of recombinant hirudin and human alpha-thrombin. Science 249, 277-280 (1990).
3 van de Locht, A. et al. Two heads are better than one: crystal structure of the insect derived double domain Kazal inhibitor rhodniin in complex with thrombin. EMBO Journal 14, 5149-5157 (1995).
4 van de Locht, A. et al. The ornithodorin-thrombin crystal structure, a key to the TAP enigma? EMBO Journal 15, 6011-6017 (1996).
5 Francischetti I M, V. J., Ribeiro J M. Anophelin: kinetics and mechanism of thrombin inhibition. Biochemistry 38, 16678-16685, doi:10.1021/bi991231p. (1999).
6 Richardson J L, K. B., Hoeffken W, Sadler J E, Pereira P, Huber R, Bode W, Fuentes-Prior P. Crystal structure of the human alpha-thrombin-haemadin complex: an exosite II-binding inhibitor. EMBO Journal 19, 5650-5660, doi:10.1093/emboj/19.21.5650. (2000).
7 Koh, C. Y. et al. Variegin, a novel fast and tight binding thrombin inhibitor from the tropical bont tick. Journal of Biological Chemistry 282, 29101-29113 (2007).
8 Macedo-Ribeiro S, A. C., Calisto B M, Friedrich T, Mentele R, Stürzebecher J, Fuentes-Prior P, Pereira P J. Isolation, cloning and structural characterisation of boophilin, a multifunctional Kunitz-type proteinase inhibitor from the cattle tick. PLoS One 3, e1624, doi:10.1371/journal.pone.0001624. (2008).
9 Wei, A. et al. Unexpected binding mode of tick anticoagulant peptide complexed to bovine factor Xa. Journal of Molecular Biology 283, 147-154 (1998).
10 Krstenansky, J. L., and Mao, Simon S. T. Antithrombin properties of C-terminus for hirudin using synthetic unsulfated N^{α}-acetyl-hirudin FEBS (Fed Eur Biochem Soc) Lett 211, 10-16, doi:10.1016/0014-5793(87)81264-4. (1987).
11 Chang, J.-Y., Schlaeppi, Jean-Marc, Stone S R. Antithrombin activity of the hirudin N-terminal core domain residues 1-43. FEBS (Fed Eur Biochem Soc) Lett 260, 209-212, doi:10.1016/0014-5793(90)80105-r (1990).
12 Krishnaswamy, S., Vlasuk, G. P. & Bergum, P. W. Assembly of the prothrombinase complex enhances the inhibition of bovine factor Xa by tick anticoagulant peptide. Biochemistry 33, 7897-7907, doi:10.1021/bi00191a017 (1994).
13 Hauel, N. H. et al. Structure-based design of novel potent nonpeptide thrombin inhibitors. J Med Chem 45, 1757-1766, doi:10.1021/jm0109513 (2002).
14 Tasset, D. M., Kubik, M. F. & Steiner, W. Oligonucleotide inhibitors of human thrombin that bind distinct epitopes. J. Mol. Biol. 272, 688-698, doi:10.1006/jmbi.1997.1275 (1997).
15 Russo Krauss, I., Pica, A., Merlino, A., Mazzarella, L. & Sica, F. Duplex-quadruplex motifs in a peculiar structural organization cooperatively contribute to thrombin binding of a DNA aptamer. Acta Crystallogr D Biol Crystallogr 69, 2403-2411, doi:10.1107/50907444913022269 (2013).
16 Alemany, A. & Ritort, F. Determination of the elastic properties of short ssDNA molecules by mechanically folding and unfolding DNA hairpins. Biopolymers 101, 1193-1199, doi:10.1002/bip.22533 (2014).
17 Baglin, T. Clinical use of new oral anticoagulant drugs: dabigatran and rivaroxaban. Br J Haematol 163, 160-167, doi:10.1111/bjh.12502 (2013).
18 Kretz, C. A., Stafford, A. R., Fredenburgh, J. C. & Weitz, J. I. HID1, a thrombin-directed aptamer, binds exosite 1 on prothrombin with high affinity and inhibits its activation by prothrombinase. J Biol Chem 281, 37477-37485, doi:10.1074/jbc.M607359200 (2006).
19 Buddai, S. K. et al. An anticoagulant RNA aptamer that inhibits proteinase-cofactor interactions within prothrombinase. J Biol Chem 285, 5212-5223, doi:10.1074/jbc.M109.049833 (2010).
20 Gunaratne, R. et al. Combination of aptamer and drug for reversible anticoagulation in cardiopulmonary bypass. Nat Biotechnol, doi:10.1038/nbt.4153 (2018).
21 Rusconi, C. P. et al. RNA aptamers as reversible antagonists of coagulation factor IXa. Nature 419, 90-94, doi:10.1038/nature00963 (2002).
22 Murphy, M. C., Rasnik, I., Cheng, W., Lohman, T. M. & Ha, T. Probing single-stranded DNA conformational flexibility using fluorescence spectroscopy. Biophys J 86, 2530-2537, doi:10.1016/S0006-3495(04)74308-8 (2004).
23 Brinkers, S., Dietrich, H. R., de Groote, F. H., Young, I. T. & Rieger, B. The persistence length of double stranded DNA determined using dark field tethered particle motion. J Chem Phys 130, 215105, doi:10.1063/1.3142699 (2009).
24 Zeng, Z., Fagnon, M., Nallan Chakravarthula, T. & Alves, N. J. Fibrin clot formation under diverse clotting conditions: Comparing turbidimetry and thromboelastography. Thromb Res 187, 48-55, doi:10.1016/j.thromres.2020.01.001 (2020).
25 Kim, Y., Cao, Z. & Tan, W. Molecular assembly for high-performance bivalent nucleic acid inhibitor. Proc Natl Acad Sci USA 105, 5664-5669, doi:10.1073/pnas.0711803105 (2008).
26 Despotis, G. J., Gravlee, G., Filos, K. & Levy, J. Anticoagulation monitoring during cardiac surgery: a review of current and emerging techniques. Anesthesiology 91, 1122-1151, doi:10.1097/00000542-199910000-00031 (1999).
27 Horton, S. & Augustin, S. Activated clotting time (ACT). Methods Mol. Biol. 992, 155-167, doi:10.1007/978-1-62703-339-8_12 (2013).
28 Tsae, P. K. & DeRosa, M. C. Outlook for aptamers after twenty five years. Curr Top Med Chem 15, 1153-1159, doi:10.2174/1568026615666150413154038 (2015).
29 Nimjee, S. M., White, R. R., Becker, R. C. & Sullenger, B. A. Aptamers as Therapeutics. Annu Rev Pharmacol Toxicol 57, 61-79, doi:10.1146/annurev-pharmtox-010716-104558 (2017).
30 Sullenger, B., Woodruff, R. & Monroe, D. M. Potent anticoagulant aptamer directed against factor IXa blocks macromolecular substrate interaction. J Biol Chem 287, 12779-12786, doi:10.1074/jbc.M111.300772 (2012).
31 Nimjee, S. M. et al. Synergistic effect of aptamers that inhibit exosites 1 and 2 on thrombin. Rna 15, 2105-2111, doi:10.1261/rna.1240109 (2009).
32 Jeter, M. L. et al. RNA aptamer to thrombin binds anion-binding exosite-2 and alters protease inhibition by heparin-binding serpins. FEBS Lett 568, 10-14, doi:10.1016/j.febslet.2004.04.087 (2004).
33 Woodruff, R. S. et al. Generation and characterization of aptamers targeting factor XIa. Thromb Res 156, 134-141, doi:10.1016/j.thromres.2017.06.015 (2017).
34 Woodruff, R. S. et al. Inhibiting the intrinsic pathway of coagulation with a factor XII-targeting RNA aptamer. J Thromb Haemost 11, 1364-1373, doi:10.1111/jth.12302 (2013).
35 Layzer, J. M. & Sullenger, B. A. Simultaneous generation of aptamers to multiple gamma-carboxyglutamic acid proteins from a focused aptamer library using DeSELEX and convergent selection. Oligonucleotides 17, 1-11, doi:10.1089/oli.2006.0059 (2007).
36 Steen Burrell, K. A., Layzer, J. & Sullenger, B. A. A kallikrein-targeting RNA aptamer inhibits the intrinsic pathway of coagulation and reduces bradykinin release. J Thromb Haemost 15, 1807-1817, doi:10.1111/jth.13760 (2017).
37 Blake, C. M., Sullenger, B. A., Lawrence, D. A. & Fortenberry, Y. M. Antimetastatic Potential of PAI-1-Specific RNA Aptamers. Oligonucleotides 19, 117-128, doi:10.1089/oli.2008.0177 (2009).
38 Damare, J., Brandal, S. & Fortenberry, Y. M. Inhibition of PAI-1 antiproteolytic activity against tPA by RNA aptamers. Nucleic Acid Ther 24, 239-249, doi:10.1089/nat.2013.0475 (2014).
39 Ren, X. et al. Evolving A RIG-I Antagonist: A Modified DNA Aptamer Mimics Viral RNA. J Mol Biol 433, 167227, doi:10.1016/j.jmb.2021.167227 (2021).
40 Li, Y. et al. Optimized Reaction Conditions for Amide Bond Formation in DNA-Encoded Combinatorial Libraries. ACS Comb Sci 18, 438-443, doi:10.1021/acscombsci.6b00058 (2016).
41 Vonrhein, C. et al. Data processing and analysis with the autoPROC toolbox. Acta Crystallogr D Biol Crystallogr 67, 293-302, doi:10.1107/S0907444911007773 (2011).
42 Kabsch, W. Xds. Acta Crystallogr D Biol Crystallogr 66, 125-132, doi:10.1107/S0907444909047337 (2010).
43 Evans, P. R. & Murshudov, G. N. How good are my data and what is the resolution? Acta Crystallogr D Biol Crystallogr 69, 1204-1214, doi:10.1107/S0907444913000061 (2013).
44 Evans, P. Scaling and assessment of data quality. Acta Crystallogr D Biol Crystallogr 62, 72-82, doi:10.1107/50907444905036693 (2006).
45 Bode, W. et al. The refined 1.9 A crystal structure of human alpha-thrombin: interaction with D-Phe-Pro-Arg chloromethylketone and significance of the Tyr-Pro-Pro-Trp insertion segent. EMBOJ8, 3467-3475, doi:10.1002/j.1460-2075.1989.tb08511.x (1989).
46 Pica, A. et al. Through-bond effects in the ternary complexes of thrombin sandwiched by two DNA aptamers. Nucleic Acids Res 45, 461-469, doi:10.1093/nar/gkw1113 (2017).
47 Liebschner, D. et al. Macromolecular structure determination using X-rays, neutrons and electrons: recent developments in Phenix. Acta Crystallogr D Struct Biol 75, 861-877, doi:10.1107/S2059798319011471 (2019).
48 Emsley, P. & Cowtan, K. Coot: model-building tools for molecular graphics. Acta Crystallogr D Biol Crystallogr 60, 2126-2132, doi:10.1107/S0907444904019158 (2004).
49 Joosten, R. P., Long, F., Murshudov, G. N. & Perrakis, A. The PDB_REDO server for macromolecular structure model optimization. IUCrJ 1, 213-220, doi:10.1107/S2052252514009324 (2014).

### Example 2—Generality for Engineering of EXACT Inhibitors

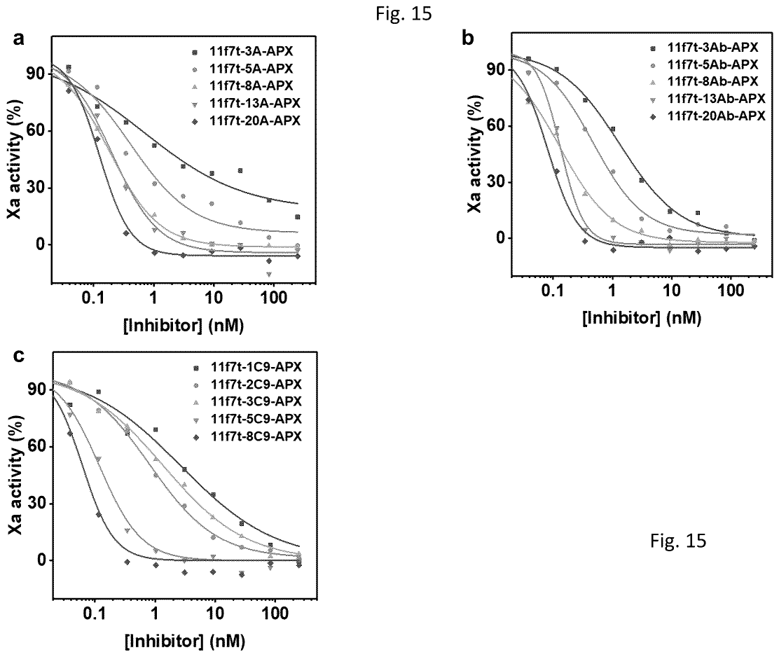

EXACT inhibitors with non-nucleic acid linkers. The inventors tested if the poly nucleic acid linker in an EXACT inhibitor can be replaced with a non-nucleic acid linker to expand the generality of inhibitor engineering. As an example, the poly 2′OMe Adenosine linker of 11F7t-APX complex (FIG. 15a) was replaced with poly deoxyribose (abasic site, FIG. 15b) or polyethylene glycol (PEG3, FIG. 15c) with varying lengths. The result showed that 11F7t-APX with non-nucleic acid linkers are able to inhibit factor Xa in similar efficiency to that with poly 2′OMe Adenosine linker.

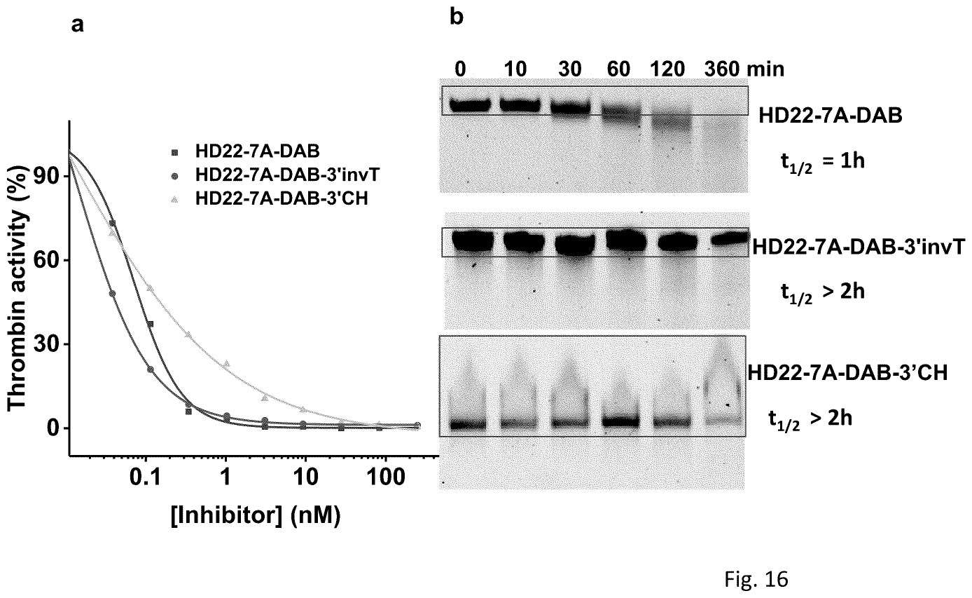

Improvement of plasma stability of HD22-7A-DAB by end protection. Plasma stability is essential for in vivo applications of EXACT inhibitors. HD22-7A-DAB showed a half-life in plasma around 1 hour (FIG. 16) due to nuclease degradation. To further improve the plasma stability of the EXACT inhibitor, the inventors modified the 3′ terminus of HD22-7A-DAB with inverted dT or cholesterol. Both variants (HD22-7A-DAB 3′invT and HD22-7A-DAB 3′CH) showed similar inhibition potency to HD22-7A-DAB (FIG. 16a) and prolongs the half-life in plasma over 2 hours (FIG. 16b).

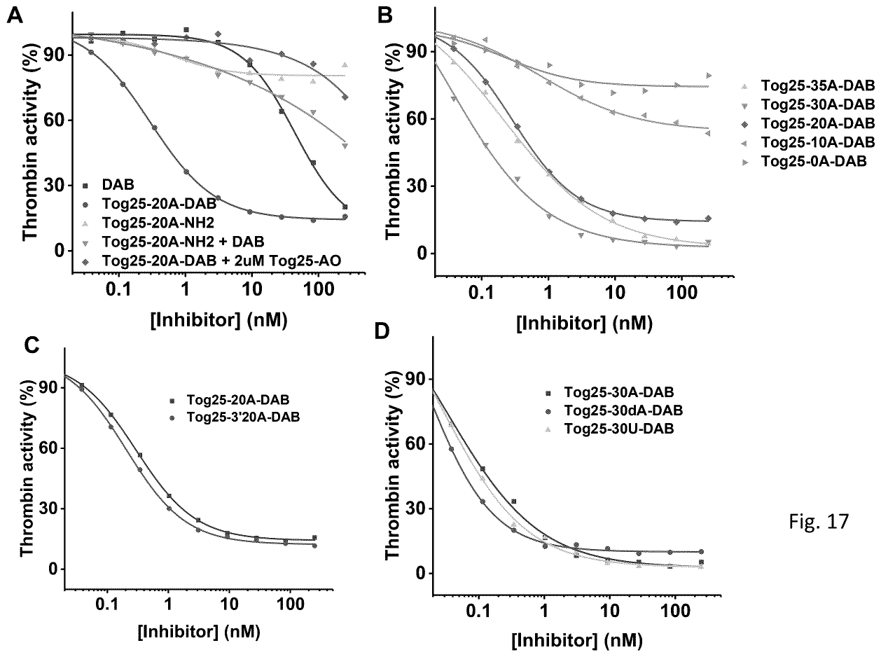

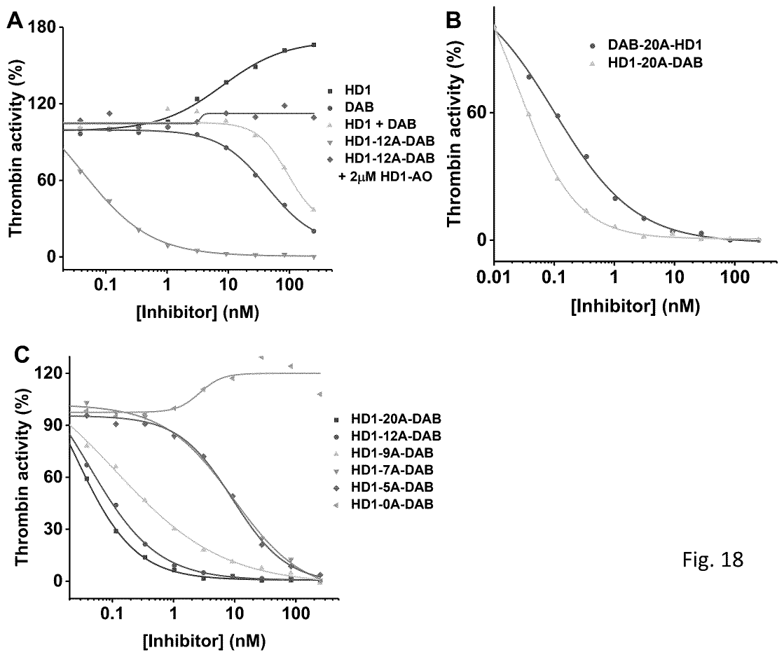

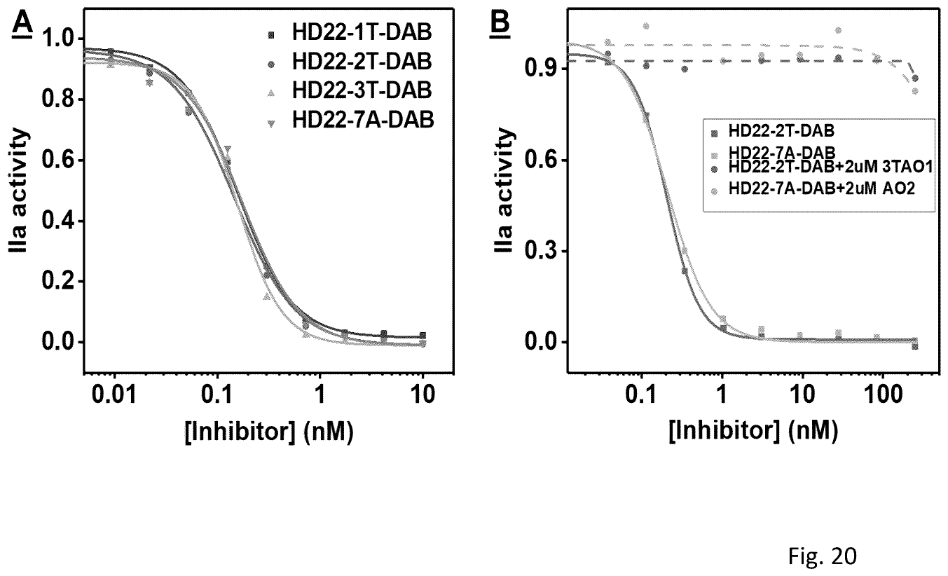

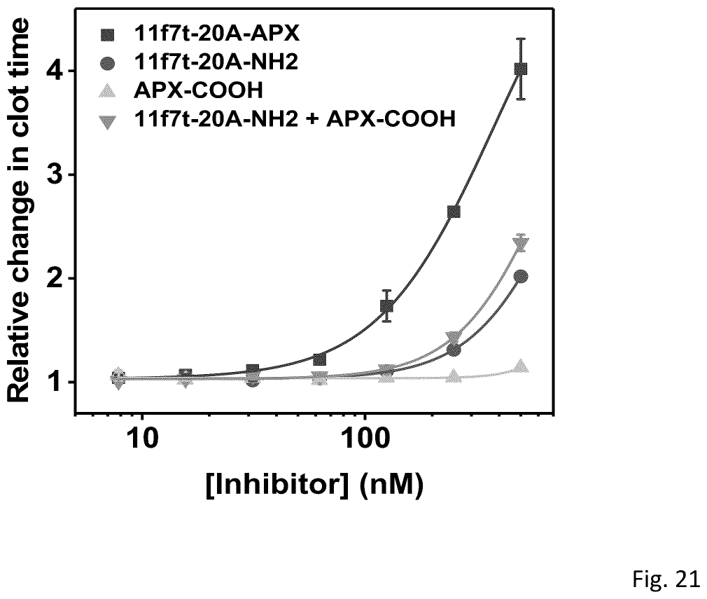

Thrombin inhibiting EXACT inhibitors engineered with different aptamers. Several thrombin-binding aptamers other than HD22 have been developed to-date. The inventors then tested if EXACT inhibitors can be readily developed using these aptamers. Specifically, the inventors developed a series of EXACT inhibitors using two aptamers, Tog25t (FIG. 17) and HD1 (FIG. 18), by conjugating DAB to either terminus of the aptamer, with different linker length and linker chemistry, and found that the optimal EXACT inhibitor generated from both aptamers are able to inhibit thrombin with similar potency to HD22-7A-DAB. EXACT inhibitors Tog25-20A-DAB and HD1-12A-DAB can also be efficiently reversed by specific antidotes (FIGS. 17 and 18). The inventors also generated HD22-DAB conjugates with the linker connected to the 3′ terminus of the aptamer which has similar potency to HD22-7A-DAB (FIG. 20A) and can be efficiently reversed by a specific antidote (FIG. 20B). These results clearly demonstrate the generality of EXACT inhibitor engineering.

1. A composition for inhibiting a protease or coagulation target comprising a nucleic acid aptamer covalently linked to a small molecule inhibitor to form an oligo-drug conjugate, wherein the nucleic acid aptamer targets the exosite of the protease or the coagulation target and wherein the small molecule inhibitor inhibits the active site of the protease or the coagulation target, wherein the nucleic acid aptamer and small molecule inhibitor bind to and inhibit a protease or coagulation target selected from the group consisting of VOFF, FIXa, FXa, XIa, XIIa, VIIa, active protein C, plasmin, prothrombin, and thrombin.

2. (canceled)

3. The composition of claim 1, wherein the nucleic acid aptamer comprises at least one of HD1, HD22, 11f7t, Tog25t, R9D-14T, 11.16, 12.7, 7S-1, 9.3T, 17-1, 7S-2, 7K-2, 7K-3, 7K-58, 7K-5, 9D-24, 10S-20, 9D-6, 9D-10, 9D15, 9D20 or 9D-31.

4. (canceled)

5. The composition of claim 1, wherein the small molecule inhibitor is a thrombin inhibitor or a factor Xa (FXa) inhibitor.

6. (canceled)

7. (canceled)

8. The composition of claim 1, wherein the composition is more potent than the aptamer or small molecular inhibitor alone or their equimolar mixture.

9. The composition claim 1, wherein the oligo-drug conjugate comprises a linker connecting the nucleic acid aptamer and the small molecule inhibitor.

10. The composition of claim 9, wherein the linker comprises 1-30 nucleotides.

11. The composition of claim 10, wherein the nucleotides comprise adenosine, uracil or thymidine or a modified adenosine, a modified uracil or a modified thymidine and wherein the modification comprises 2′-OMe or 2′-Fluro and invdT.

12. (canceled)

13. (canceled)

14. The composition of claim 9, wherein the linker is a non-nucleotide linker, and optionally wherein the non-nucleotide linker comprises, a basic site, or polyethylene glycol.

15. The composition of claim 9, wherein the linker is attached to the 5′ end or the 3′ end of the aptamer.

16. (canceled)

17. (canceled)

18. The composition of claim 1, wherein,
the nucleic acid aptamer is HD22 and the coagulation inhibitor is dabigatran; or
the nucleic acid aptamer is 11F7t and the coagulation inhibitor is apixaban or dabigatran; or
the nucleic acid aptamer is Tog25 and the coagulation inhibitor is dabigatran; or
the nucleic acid aptamer is HD1 and the coagulation inhibitor is dabigatran.

19.-21. (canceled)

22. The composition of claim 18, wherein the linker is seven to thirty adenosine nucleotides.

23. (canceled)

24. (canceled)

25. A method of inhibiting coagulation comprising contacting a site of coagulation or potential coagulation with the composition of claim 1.

26. The method of claim 25, further comprising administering an antidote to the composition, wherein the antidote comprises a complementary oligonucleotide to a portion of the aptamer and linker and inhibits the binding of the composition to its target.

27. The method of claim 25, wherein the composition is administered by injection.

28. (canceled)

29. The method of claim 25, wherein the method prevents or treats thrombosis, or is for use before, during or after cardiopulmonary bypass surgery or percutaneous coronary intervention.

30.-32. (canceled)

33. A method of treating a subject in need of anti-coagulation therapy, the method comprising:
administering the composition of claim 1 to the subject for a period of time sufficient to allow a reduction in coagulation in the subject, and
administering an antidote, wherein the antidote comprises an oligonucleotide complementary to at least a portion of the aptamer and reverses the anti-coagulation activity of the composition and wherein the composition is administered by injection.

34.-36. (canceled)

37. The method according to claim 33, wherein the method prevents or treats thrombosis.

38. The method according to claim 33, wherein the subject is a surgery patient or wherein the subject is undergoing cardiopulmonary bypass surgery or percutaneous coronary intervention or wherein the subject suffers from stroke or deep vein thrombosis.

39. (canceled)

40. (canceled)

41. A method of increasing the affinity of a coagulation inhibitor for a target, the method comprising conjugating a coagulation inhibitor with weak affinity for a target to an aptamer specific for the target with a linker.

42. The method of claim 41, wherein the aptamer is 11F7t and the coagulation inhibitor is dabigatran.
